(19) United States
(12) Patent Application Publication
(10) Pub. No.: US 2024/0240189 A1
(43) Pub. Date: Jul. 18, 2024

(54) CANCER TREATMENT BY TARGETING PROTEINS OR INTERACTIONS OF EPHRINB-RGS3-KIF20A-SEPT7 AXIS

(71) Applicant: CITY OF HOPE, Duarte, CA (US)

(72) Inventors: Qiang LU, Duarte, CA (US); Runxiang QIU, Duarte, CA (US)

(73) Assignee: CITY OF HOPE, Duarte, CA (US)

(21) Appl. No.: 18/554,286

(22) PCT Filed: Apr. 6, 2022
(86) PCT No.: PCT/US22/23737

§ 371 Date: Oct. 6, 2023

Related U.S. Application Data
(60) Provisional application No. 63/172,403, filed on Apr. 8, 2021.

(51) Int. Cl. C12N 15/113; A61K 38/08; A61K 38/17; A61P 35/00

(52) U.S. Cl. CPC C12N 15/1138; A61K 38/08; A61K 38/1709; A61P 35/00; C12N 2310/141; C12N 2310/531

(57) ABSTRACT

A method of treating cancer in a subject by targeting at least one of KIF20A, SEPT7, RGS3 and EphrinB or disrupting protein-protein interaction in the EPHRINB-RGS3-KIF20A-SEPT7 axis. The method entails administering to the subject an RNA-based inhibitor such as an siRNA, shRNA, or miRNA or a peptide inhibitor which targets at least one of KIF20A, SEPT7, RGS3 and EphrinB or blocks the binding of KID20A to RGS3 and/or SEPT7 or the binding of EphrinB and RGS3.

### PRIORITY CLAIM

This application claims priority to U.S. Provisional Patent Application No. 63/172,403, filed Apr. 8, 2021, the contents of which are hereby incorporated by reference in its entirety.

### STATEMENT REGARDING FEDERALLY SPONSORED RESEARCH

This invention was made with government support under Grant Number NS096130, awarded by the National Institutes of Health. The government has certain rights in the invention.

### SEQUENCE LISTING

This application contains a Sequence Listing, which was submitted in ASCII format via EFS-Web, and is hereby incorporated by reference in its entirety. The ASCII copy, created on Apr. 6, 2022, is named SequenceListing.txt and is 48 KB in size.

### BACKGROUND

Most mitosis inhibitors used in chemotherapy for brain or other types of tumors act to disrupt cell divisions. Such inhibitors could often lead to an unintended effect of creating multinucleated cells or aneuploidy, which can be highly mutagenic and produce mutations that become resistant to the original inhibitors. Accumulating evidence has demonstrated that various cancers such as brain tumors are often originated from dysregulated neural stem or progenitor cells, therefore, molecules that are involved in regulating the proliferative properties of normal neural stem/progenitor cells may serve as potential therapeutic targets for developing anti-proliferation therapy. However, such molecules in normal neural stem cells, particularly those acting during the time of cell divisions, have remained largely elusive. Accordingly, this disclosure satisfies the need in the art by providing a novel cancer therapy.

### SUMMARY

In one aspect, disclosed herein is a method of treating cancer in a subject by targeting a protein or disrupting protein-protein interaction in the EPHRINB-RGS3-KIF20A-SEPT7 axis, thereby to inhibit proliferation of a daughter cell produced by a parental stem cell, progenitor cell, or cancer cell. The method comprises administering to the subject an effective amount of an RNA-based inhibitor or a peptide inhibitor which targets at least one of KIF20A, SEPT7, RGS3 and EphrinB or prevents or blocks binding of KIF20A to RGS3 or to SEPT7 or binding of EphrinB to RGS3. In some embodiments, the RNA-based inhibitor is an siRNA, an shRNA, or a miRNA targeting KIF20A, SEPT7, RGS3 or EphrinB. In certain embodiments, the peptide inhibitor is a peptide comprising an amino acid sequence at least 75%, at least 80%, at least 85%, at least 90%, at least 95%, at least 98%, at least 99%, or 100% identical to the amino acid sequence of mouse KIF20A residues 625-818 or human KIF20A residues 626-820. In certain embodiments, the peptide inhibitor is a fragment of mouse KIF20A₆₂₅₋₈₁₈ or human KIF20A₆₂₆₋₈₂₀. In certain embodiments, the peptide inhibitor is a small peptide comprising 6-9 amino acid residues such as 6, 7, 8, or 9 amino acids of the C-terminus of the conserved EphrinB proteins. In some embodiments, the small peptide comprises an amino acid sequence at least 75%, at least 80%, at least 85%, at least 90%, at least 95%, at least 98%, at least 99%, or 100% identical to the amino acid sequence of SPANIYYKV (SEQ ID NO: 1). In some embodiments, the small peptide comprises an amino acid sequence at least 75%, at least 80%, at least 85%, at least 90%, at least 95%, at least 98%, at least 99%, or 100% identical to the amino acid sequence of ANIYYKV (SEQ ID NO: 2). In some embodiments, the small peptide comprises an amino acid sequence at least 75%, at least 80%, at least 85%, at least 90%, at least 95%, at least 98%, at least 99%, or 100% identical to the amino acid sequence of NIYYKV (SEQ ID NO: 3). In certain embodiments, the peptide inhibitor is a peptide comprising an amino acid sequence at least 75%, at least 80%, at least 85%, at least 90%, at least 95%, at least 98%, at least 99%, or 100% identical to the amino acid sequence of the PDZ domain of mouse or human RGS3 isoform 1 (PDZ-RGS3) residues 18-94. In some embodiments, the cancer includes brain tumor, leukemia, breast cancer, lung cancer, colon cancer, and liver cancer. In some embodiments, the brain tumor is a malignant brain tumor such as medulloblastoma (MB) or glioblastoma (GBM).

In another aspect, disclosed is a method of inhibiting proliferation of a daughter cell produced by a cancer cell in a subject. The method comprises administering to the subject an effective amount of an RNA-based inhibitor or a peptide inhibitor which targets at least one of KIF20A, SEPT7, RGS3 and EphrinB or prevents or blocks binding of KIF20A to RGS3 or to SEPT7 or binding of EphrinB to RGS3. In some embodiments, the RNA-based inhibitor is an siRNA, an shRNA, or a miRNA targeting KIF20A, SEPT7, RGS3 or EphrinB. In certain embodiments, the peptide inhibitor is a peptide comprising an amino acid sequence at least 75%, at least 80%, at least 85%, at least 90%, at least 95%, at least 98%, at least 99%, or 100% identical to the amino acid sequence of mouse KIF20A residues 625-818 or human KIF20A residues 626-820. In certain embodiments, the peptide inhibitor is a fragment of mouse KIF20A₆₂₅₋₈₁₈ or human KIF20A₆₂₆₋₈₂₀. In certain embodiments, the peptide inhibitor is a small peptide comprising 33 amino acid residues or less of the C-terminus of the conserved EphrinB proteins. In certain embodiments, the peptide inhibitor is a small peptide comprising 6-9 amino acid residues such as 6, 7, 8, or 9 amino acids of the C-terminus of the conserved EphrinB proteins. In some embodiments, the small peptide comprises an amino acid sequence at least 75%, at least 80%, at least 85%, at least 90%, at least 95%, at least 98%, at least 99%, or 100% identical to the amino acid sequence of SPANIYYKV (SEQ ID NO: 1). In some embodiments, the small peptide comprises an amino acid sequence at least 75%, at least 80%, at least 85%, at least 90%, at least 95%, at least 98%, at least 99%, or 100% identical to the amino acid sequence of ANIYYKV (SEQ ID NO: 2). In some embodiments, the small peptide comprises an amino acid sequence at least 75%, at least 80%, at least 85%, at least 90%, at least 95%, at least 98%, at least 99%, or 100% identical to the amino acid sequence of NIYYKV (SEQ ID NO: 3). In certain embodiments, the peptide inhibitor is a peptide comprising an amino acid sequence at least 75%, at least 80%, at least 85%, at least 90%, at least 95%, at least 98%, at least 99%, or 100% identical to the amino acid sequence of the PDZ domain of mouse or human RGS3 isoform 1 (PDZ-RGS3) residues18-94. In some embodiments, the cancer includes brain tumor, leukemia, breast cancer, lung cancer, colon cancer, and liver cancer. In some embodiments, the brain tumor is a malignant brain tumor such as medulloblastoma or glioblastoma.

In another aspect, disclosed is a composition for treating cancer comprising an siRNA, an shRNA, or a miRNA targeting KIF20A, SEPT7, RGS3 or EphrinB, a peptide comprising an amino acid sequence at least 75%, at least 80%, at least 85%, at least 90%, at least 95%, at least 98%, at least 99%, or 100% identical to the amino acid sequence of mouse KIF20A residues 625-818 or human KIF20A residues 626-820, or a fragment thereof, or a small peptide comprising 33 amino acid residues or less of the C-terminus of the conserved EphrinB proteins. In certain embodiments, the peptide inhibitor is a small peptide comprising 6-9 amino acid residues such as 6, 7, 8, or 9 amino acids of the C-terminus of the conserved EphrinB proteins. In some embodiments, the small peptide comprises an amino acid sequence at least 75%, at least 80%, at least 85%, at least 90%, at least 95%, at least 98%, at least 99%, or 100% identical to the amino acid sequence of SPANIYYKV (SEQ ID NO: 1). In some embodiments, the small peptide comprises an amino acid sequence at least 75%, at least 80%, at least 85%, at least 90%, at least 95%, at least 98%, at least 99%, or 100% identical to the amino acid sequence of ANIYYKV (SEQ ID NO: 2). In some embodiments, the small peptide comprises an amino acid sequence at least 75%, at least 80%, at least 85%, at least 90%, at least 95%, at least 98%, at least 99%, or 100% identical to the amino acid sequence of NIYYKV (SEQ ID NO: 3). In certain embodiments, the peptide inhibitor is a peptide comprising an amino acid sequence at least 75%, at least 80%, at least 85%, at least 90%, at least 95%, at least 98%, at least 99%, or 100% identical to the amino acid sequence of the PDZ domain of mouse or human RGS3 isoform 1 (PDZ-RGS3) residues 18-94. In certain embodiments, the siRNA, shRNA, miRNA or the peptide is conjugated to a delivery vehicle such as a nanoparticle. In certain embodiments, the composition further comprising one or more pharmaceutically acceptable excipients or carriers. In some embodiments, the cancer includes brain tumor, leukemia, breast cancer, lung cancer, colon cancer, and liver cancer. In some embodiments, the brain tumor is a malignant brain tumor such as medulloblastoma or glioblastoma.

### BRIEF DESCRIPTION OF THE DRAWINGS

This application contains at least one drawing executed in color. Copies of this application with color drawing(s) will be provided by the Office upon request and payment of the necessary fees.

FIGS. 1a-1d show that loss of function (LOF) of KIF20A in granule neuron progenitors (GNPs) resulted in a loss of proliferating cells. FIG. 1a shows that KIF20A expression was mainly enriched in the proliferating cell zone in the external granular layer (EGL) of the early postnatal cerebellum. Scale bar represents 50 μm. FIG. 1b shows that Tamoxifen (TM)-induced knockout of Kif20a in GNPs caused a reduction in the number of proliferating cells in the mutant cerebellums. Ki67⁺ cells within boxed region at the center of individual cerebellar sulcus were used for quantification. White arrowheads indicated the tips of sulci in the mutant cerebellums, which often had diminished Ki67 signal. Scale bar represents 50 μm. Data are mean±S.D. P=0.0019 (P5), 6.65E-05 (P6), 9.52E-05 (P7) (Student's t-test). FIG. 1c shows that knockout of Kif20a did not cause noticeable increase of apoptosis level marked by cleaved caspase 3 in the P6 mutant cerebellums. Scale bar represents 50 μm. Data are mean±S.D. FIG. 1d shows that flow cytometry-based cell cycle analysis of P6 littermate of knockout and control cerebellums. The 4N DNA contents (representing G2/M phase cells or cells with two nuclei resulted from cytokinesis defect) were little changed between the Kif20a knockout and control brains. n.s. represents not significant.

FIGS. 2a-2c show analyses of proliferation and apoptosis status in the Kif20a knockout cerebellum. FIG. 2a shows staining of KIF20A in P5 and P6 control and Kif20a knockout cerebellums. Tamoxifen was given to pups at P4. Scale bar represents 50 μm. FIG. 2b shows staining of proliferation marker Ki67 in P14 control and Kif20a knockout cerebellums. Tamoxifen was given to pups at P4. Data are mean±S.D. ***P<0.001 (student's t-test). FIG. 2c shows knockout of Kif20a did not cause noticeable change of apoptosis level marked by cleaved caspase 3 in the mutant cerebellum (P7). Scale bar represents 50 μm. Data are mean±S.D. n.s. represents not significant.

FIGS. 3a-3c show LOF of KIF20A in GNPs caused early cell cycle exit and precocious neuronal differentiation. FIG. 3a shows that after tamoxifen (TM) treatment at P4, animal pups were labeled with EdU at P5 and then brains were collected at P6. Co-staining of EdU and Ki67 showed that knockout of Kif20a resulted in relatively more EdU+Ki67− cells in the EdU+ cell population compared to the wild-type littermate brains. Scale bar represents 50 μm. Data are mean±S.D. P=1.09E-09 (Student's t-test). FIG. 3b shows that more EdU+Ki67⁻ cells in the mutant cerebellums were positive for neuronal marker NeuN (white arrowheads). These cells formed a line outside the Ki67⁺ cells, reflecting they were differentiating and migrating out of the EGL. Scale bar represents 50 μm. Data are mean±S.D. P=1.29E-06 (Student's t-test). FIG. 3c shows that after tamoxifen treatment at P4, animal pups were labeled with EdU at P5 and then brains were collected at P7. Co-staining of EdU and Ki67 showed that knockout of Kif20a resulted in fewer EdU⁺Ki67⁺ proliferating cells in the EGL at this stage. EdU⁺Ki67⁺ cells within individual cerebellar sulcus were used for quantification. Scale bar represents 50 μm. Data are mean±S.D. P=1.58E-07 (Student's t-test).

FIGS. 4a-4h show that LOF of KIF20A in tumor-initiating GNPs inhibited SHH-induced medulloblastoma (MB) formation. FIG. 4a shows that single (Atoh1-CreER; Ptc^{fl/fl}) and double (Atoh1-CreER; Ptc^{fl/fl}; Kif20a^{fl/fl}) knockout mice were treated with tamoxifen at P4 by gavage. The mice were euthanized when brain tumor symptoms were developed and brains were collected for analyses. Survival of mice was summarized in the Kaplan-Meier curve. FIG. 4b shows representative whole brain samples from wild-type (Ptc^{fl/fl}), Ptc single (Atoh1-CreER; Ptc^{fl/fl}), or Ptc/Kif20a double (Atoh1-CreER; Ptc^{fl/fl}; Kif20a^{fl/fl}) knockout mice. The numbers in each panel indicate the days when brain samples were collected after TM injection (sections of these brains were shown in 4c-4h). FIG. 4c shows normal brain features from section of wild-type control (Ptc^{fl/fl}) brain sacrificed 145 days after TM injection. Arrowhead indicates the cerebellum. FIGS. 4d and 4e show that examples of sections from Ptc single knockout mice displayed strong tumor growth and often disformed brain structures. Arrows indicate the tumor mass. FIGS. 4f-4h show that examples of sections from Ptc/Kif20a double mice displayed varied tumor sizes. The overall brain structures were in general in better shape than the single knockout mice.

FIGS. 5a-5c show that inducible knockout of Kif20a in tumor-initiating GNPs caused early cell cycle exit. FIG. 5a shows that after tamoxifen treatment at P4, animal pups were labeled with EdU at P5 and then brains were collected at P6. Co-staining of EdU and Ki67 revealed that knockout of Kif20a in MB-initiating GNPs (Ptc single knockout GNPs) resulted in more EdU⁺Ki67⁻ cells in the EdU+ cell population. White arrowheads indicate the EdU⁺Ki67⁻ cells, many of which line outside the proliferating cell zone. Scale bar represents 50 μm. Quantifications from FIG. 3a (green and red columns) were plotted together in the graph. Data are mean±S.D. P=1.82E-05 (between the first two columns) (Student's t-test). FIG. 5b shows that more EdU⁺Ki67⁻ cells in the Ptc/Kif20a double knockout cerebellums were positive for neuronal marker NeuN (white arrowheads). Scale bar represents 50 μm. Data are mean±S.D. P=0.00013 (Student's t-test). FIG. 5c shows that after tamoxifen treatment at P4, animal pups were labeled with EdU at P5 and then brains were collected at P7. Co-staining of EdU and Ki67 shows that knockout of Kif20a in MB-initiating GNPs resulted in fewer EdU⁺Ki67⁺ proliferating cells in the EGL. EdU⁺Ki67⁺ cells within individual cerebellar sulcus were used for quantification. Scale bar represents 50 μm. Data are mean±S.D. P=0.00042 (Student's t-test).

FIGS. 6a-6d show generation of tumor cell lines from Ptc single and Ptc/Kif20a double knockout mice. FIG. 6a is a schematic illustration of PCR strategies for genotyping Kif20a and Ptc knockout in mouse tumor cells. FIGS. 6b and 6c show that primers of P1, P2 and P3 were combined for PCR on genomic DNAs. Tumor cells from both the single (Atoh1-CreER; Ptc^{fl/fl}) and double (Atoh1-CreER; Ptc^{fl/fl+}; Kif20^{fl/fl}) knockout mice showed homozygous deletion of Ptc. However, tumor cells from the double knockout mice were found to be heterozygous for Kif20a. *indicates the lane showing heterozygous bands of Kif20a alleles. FIG. 6d shows that qPCR on RNAs isolated from tumor cells derived from the Ptc and Kif20a double knockout mice demonstrated reduced level of Kif20a mRNA. ***P<0.001 (Student's t-test).

FIGS. 7a-7d show that LOF of KIF20A in mouse tumor cells inhibited proliferation by inducing cell cycle exit. FIG. 7a shows that two tumor cell lines were derived from the Ptc single knockout mice and these cells carried a genotype of Ptc^{-/-}; Kif20a^{+/+}. Two tumor cell lines from the Ptc/Kif20a double knockout mice were also established in culture and these cells showed a genotype of Ptc^{-/-}; Kif20a^{fl/-}. The latter two tumor lines displayed slower proliferation rates. Individual data points represented replicates of cell samples. Data are mean±S.D. P=9.46E-05 (two-way ANOVA). FIG. 7b shows that proliferating tumor cells were labeled with EdU for 24 hours and were then stained for EdU and Ki67. There were fewer Ki67⁺ cells and a relatively high percentage of EdU⁺Ki67⁻ cells (in the total population of EdU+ cells) in the tumor line derived from the Ptc/Kif20a double knockout mice. Scale bar represents 50 μm. Data are mean±S.D. P=5.45E-08; 1.75E-07 (Student's t-test). FIG. 7c shows that tumor cells having a genotype of Ptc^{-/-}; Kif20a^{fl/-} derived from the Ptc/Kif20a double knockout mice were infected with lentivirus expressing Cre-2A-GFP or control GFP and were next labeled with EdU for 24 hours in culture. Knockout of the remaining Kif20a allele in these cells resulted in cell cycle exit. Scale bar represents 50 μm. Data are mean±S.D. P=4.12E-05 (Student's t-test). FIG. 7d shows that tumor cells derived from the Ptc/Kif20a double knockout mice were infected with lentivirus expressing Cre-2A-GFP or control GFP. After labeling with propidium iodide, the cells were examined for their DNA contents by flow cytometry analysis. PI, propidium iodide; 2N, cells in G1 phase; 4N, cells in G2/M phase or bi-nucleated cells. n.s. represents not significant.

FIG. 8 shows FACS-based cell cycle analysis of SHH-MB tumor cell lines. Tumor cells derived from the Ptc single and Ptc/Kif20a double knockout mice were labeled with propidium iodide and their DNA profiles were examined by flow cytometry. PI, propidium iodide; 2N, cells in G1 phase; 4N, cells in G2/M phase or bi-nucleated cells. n.s. represents not significant.

FIGS. 9a-9e show that knockdown of KIF20A expression in human MB cells inhibited tumor growth. FIG. 9a shows that different subgroups of MB patient cells displayed strong expression of KIF20A, which is positively correlated with proliferating cell marker Ki67. FIG. 9b shows that KIF20A and Ki67 expressions were also positively correlated in different subtypes of human MB cells. FIG. 9c shows comparison between KIF20A expression and Ki67 expression in subgroups of human MB cells. Expression of the two factors was highly correlated. FIG. 9d shows that Daoy cells stably integrated with Tet-on shKIF20A726 and firefly luciferase were intracranially injected into the cerebellar areas of recipient NSG mice (10⁵ cells per mouse). Tumor growths were monitored by bioluminescence imaging. Doxycycline treatment started at four weeks after initial cell implantation (indicated with a red font and line). The treatment group of mice was first given doxycycline by gavage for two consecutive days and was then fed with doxycycline-containing food for the entire duration of the experiment. FIG. 9e shows survival data analyzed by Kaplan-Meier plot.

FIGS. 10a-10c show generation of human KIF20A shRNAs and stable Daoy cell lines. FIG. 10a shows screening of shRNAs by luciferase assay. Candidate shRNAs in pNUT vector were first screened with a cDNA target cloned in psi-CHECK vector in transfected HEK293 cells, using a dual luciferase reporter assay and their cDNA target in psi-CHECK Vector (Promega). Six candidate shKIF20As were tested with this assay. Two of the shRNAs, shKIF20A181 and shKIF20A726, were found to significantly inhibit expression of the human KIF20A transcript. shKIF20A726 was further used to isolate stable clones of Daoy cells. Data are mean±S.D. FIG. 10b shows that three Daoy cell lines stably integrated with Tet-on inducible shKIF20A726 expression cassette were established. In each cell line, doxycycline (Dox) treatment led to reduction of KIF20A expression. α-Tubulin was used as a loading control. FIG. 10c shows that inducible expression of shKIF20A726 (+Dox) in each cell line was able to inhibit cell proliferation. As a technical control, parental Daoy cells showed similar growth property with or without doxycycline treatment. Data are mean±S.D. **P<0.01, ***P<0.001 (two-way ANOVA).

FIGS. 11a-121 show that knockdown of KIF20A promoted cell cycle exit and inhibited proliferation of patient-derived GBM-initiating/stem cells (GISCs). FIG. 11a: KIF20A shRNA resulted in significant knockdown of KIF20A expression in GISC017 cells. FIG. 11b: Expression of shKIF20A726 in GISC017 cells caused an increase of GFP+EdU+Ki67− cells (cells left the cell cycle) in the total population of GFP+EdU+ cells. **P<0.01 (Student's t-test). FIG. 11c: Knockdown of KIF20A in GISC017 cells inhibited cell growth. **P<0.01 (Two-way ANOVA). FIG. 11d: Knockdown of KIF20A in GISC017 cells did not cause obvious change in cytokinesis.

FIGS. 12a-12b show that inducible Kif20a knockdown in GISC017 cells suppressed cell proliferation in growing tumors. FIG. 12a: NSG mice were injected with GISC017 cells stably integrated with Tet-inducible shRNA and luciferase. Treatment with doxycycline started at 10 days after cell implantation (indicated by the red line). FIG. 12b: Survival data of the mice were analyzed by Kaplan-Meier plot.

FIGS. 13a-13c show that competitive inhibition of RGS3-KIF20A interaction promoted cell cycle exit. FIG. 13a: The RGS-binding domain (RBD) was mapped to amino acids 625-818 of mouse KIF20A. FIG. 13b: Expression of RBD inhibited binding between RGS3 and mouse KIF20A (GST-RGS pull down assay) in a dose-dependent manner. FIG. 13c: Lentiviral expression of mouse KIF20A RBD in GISC017 cells caused cell cycle exit.

FIG. 14 shows the functional test of mouse RBD (KIF20A₆₂₅₋₈₁₈) and RGS domain in the cortex. Expression plasmid of GFP, RBD (RGS-binding domain of mouse KIF20A), or RGS was delivered into the cortices at E13.5 via in utero electroporation (IUE). Brains were collected at E15.5 for analyses. Distribution of transfected cells (GFP+) across the radial domains were scored. Results show that expression of RBD (mouse KIF20A₆₂₅₋₈₁₈) caused neural progenitor cell differentiation and subsequent outward migration of newly born neurons into the IZ and CP regions. VZ, ventricular zone; IZ, intermediate zone; CP, cortical plate. Green and blue asterisks indicate P<0.01 (Student's t-test) for CP and SVZ/VZ distributions, respectively.

FIGS. 15a-15c show that competitive inhibition of EphrinB-RGS3 interaction promoted cell cycle exit and inhibited proliferation of brain tumor stem cells. FIG. 15a: Sequence alignment showed the conserved sequence of C-terminal 33 amino acids of all EphrinBs. The peptide motif required for interaction with the PDZ domain of RGS3 was located within the last 5-6 amino acids. FIG. 15b: Lentiviral expression of the C-terminal 33 amino acids of EphrinB (as a GFP-fusion protein, GFP-EfnB33) or the PDZ domain of RGS3 (as a GFP-PDZ fusion) in patient-derived glioblastoma stem cells caused cell cycle exit. ***P<0.001. FIG. 15c: Lentiviral expression of GFP-PDZ in patient-derived glioblastoma stem cells inhibited cell proliferation. ***P<0.001.

FIG. 16 shows the amino acid sequence alignment of mouse KIF20A₆₂₅₋₈₁₈ (Query) to human KIF20A (Sbjct).

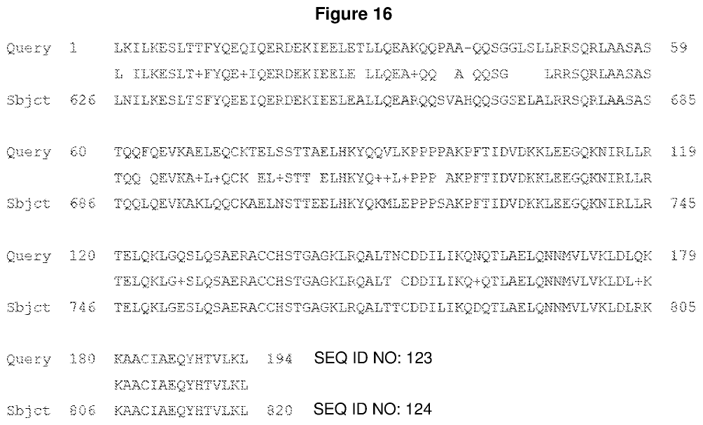

FIG. 17 illustrates an amino acid sequence alignment to show the conserved ephrinB cytoplasmic domain between EphrinB1 and EphrinB2.

FIG. 18 illustrates an amino acid sequence alignment to show the human vs. mouse PDZ domain.

### DETAILED DESCRIPTION

Disclosed herein is a method of treating cancer in a subject by disrupting protein-protein interaction in the EPHRINB-RGS3-KIF20A-SEPT7 axis, thereby to inhibit proliferation of a daughter cell produced by a parental stem cell, progenitor cell, or cancer cell. The method comprises administering to the subject an effective amount of an RNA-based inhibitor such as an siRNA, shRNA or miRNA targeting KIF20A, SEPT7, RGS3 or EphrinB, or a peptide inhibitor which prevents or blocks the binding of KIF20A to RGS3 or to SEPT7 or the binding of EphrinB and RGS3.

“Treating” or “treatment” of a disease or a condition may refer to preventing the disease or condition, slowing the onset or rate of development of the disease or condition, reducing the risk of developing the disease or condition, preventing or delaying the development of symptoms associated with the disease or condition, reducing or ending symptoms associated with the disease or condition, generating a complete or partial regression of the disease or condition, or some combinations thereof.

As used herein, the term “subject” refers to a mammalian subject, preferably a human. The phrases “subject” and “patient” are used interchangeably herein.

An “effective amount,” “therapeutically effective amount” or “effective dose” is an amount of an RNA-based inhibitor or peptide or a composition comprising an RNA-based inhibitor or peptide that produces a desired therapeutic effect in a subject, such as preventing or treating a target disease or condition, or alleviating symptoms associated with the disease or condition. The precise therapeutically effective amount is an amount of the RNA, peptide, or composition that will yield the most effective results in terms of efficacy of treatment in a given subject. This amount will vary depending upon a variety of factors, including but not limited to the characteristics of the active agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of an active agent and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, P A, 2005.

A protein-protein interaction (PPI) network in normal and cancerous neural stem cells is identified and it can serve as a novel target for developing anti-proliferation therapy for treating various cancers such as malignant brain tumors, including medulloblastoma and glioblastoma (GBM). As disclosed herein, targeting this PPI network by RNA interference using shRNAs, miRNAs or siRNAs, or by competitive inhibition using peptides comprising sequences from the protein binding domains can suppress tumor cell proliferation. These shRNAs, miRNAs, siRNAs and peptide inhibitors present the potential novel therapeutics.

This disclosure identifies some key factors that are responsible for regulating cell fate determination, the process in which the two daughter cells produced by a parental neural stem cell make their choices of either staying as a proliferating stem cell or becoming differentiated into a mature cell type. These studies uncovered a protein-protein interaction (PPI) network, including EphrinB⁴⁵, RGS3⁴⁶, KIF20A⁹, and SEPT7¹⁰, that is crucially required for maintaining the proliferative fate of a neural stem cell's daughter cells. Since the EPHRINB-RGS3-KIF20A-SEPT7 axis proteins act during the process of cell fate determination, which is downstream of most, if not all oncogenic pathways, targeting this axis of proteins can universally suppress the growth of different types or subtypes of cancers. More specifically, KIF20A and SEPT7 are mitotic proteins expressed by all proliferating cells including stem/progenitor cells and cancer cells and have been implicated in the regulation of cytokinesis of cancer cells. Thus, targeting the EPHRINB-RGS3-KIF20A-SEPT7 axis proteins can inhibit tumor growth in various cancer types, such as brain tumor, leukemia, breast cancer, lung cancer, colon cancer, liver cancer, etc. Accordingly, targeting the EPHRINB-RGS3-KIF20A-SEPT7 axis presents a novel strategy for expanding the repertoire of anti-proliferation chemotherapy for a broad spectrum of tumor types.

As demonstrated in the working examples, targeting one component of this network such as KIF20A for inhibition by specific shRNAs in mouse and human medulloblastoma cells was able to suppress tumor growth⁴⁷. Also demonstrated by an ongoing study is that inhibition of KIF20A by shRNAs or by a competitive peptide inhibitor which blocks the binding between RGS3 and KIF20A or between EphrinB and RGS3 in patient-derived glioblastoma cells was able to suppress glioblastoma tumor growth. Accordingly, this disclosure also relates to shRNAs, miRNAs, and siRNAs targeting KIF20A, RGS3, SEPT7 or EphrinB, as well as competitive peptide inhibitors, for treating cancer such as malignant brain tumors and leukemia. In certain embodiments, disclosed herein is an RNA-based inhibitor or a peptide inhibitor for KIF20A that inhibits or blocks the binding between RGS3 and KIF20A or between KIF20A and SEPT7.

Current anti-proliferation chemotherapies use mitosis inhibitors that are known to disrupt cell divisions. As demonstrated by the previous publications^{9-10, 45-47} and additional data disclosed herein, targeting cell fate determination process by inhibiting the EPHRINB-RGS3-KIF20A-SEPT7 axis does not block mitosis per se, but rather causing the daughter cells of a parental stem cell to become post-mitotic cells thereby reducing the proliferating cell pool. This inhibitory strategy presents a more effective inhibition of proliferation with less toxicity. The EPHRINB-RGS3-KIF20A-SEPT7 axis is the first pathway of proteins identified to control the decision of the fate of the daughter cells from a proliferating cell such as a stem/progenitor cell or a tumor cell to either stay as proliferating cells or become post-mitotic cells. The data show that interfering with the function of one component of this axis, KIF20A, in malignant brain tumor cells was able to drive the cells to differentiate into mature cells. Thus, targeting this axis of proteins or their interactions does not disrupt cell division, but rather induces the newly generated daughter cells to exit the proliferation route.

Various RNA-based inhibitors and peptide inhibitors can be used in the disclosed technology. For example, RNA-based inhibitors that can block interactions between KIF20A and RGS3 or SEPT7 or between EphrinB and RGS3. Inhibitors of KIF20A-RGS3 interaction can be isolated by solving the structure of KIF20A-RGS3 complex via X-ray crystallography, and then identify candidate inhibitor molecules based on the crystallography information. In certain embodiments, siRNA-based, shRNA-based, or miRNA-based KIF20A, RGS3, SEPT7 or EphrinB inhibitors can be used to inhibit cancer cell proliferation. The siRNAs, shRNAs, and the miRNAs can be delivered to a subject via conjugates, e.g., conjugated to nanoparticles or aptamers.

The shRNAs are developed using a web-based designing tool, which predicts potential shRNA sequences within cDNAs encompassing the coding sequence (CDS) and/or 3′ untranslated region (3′ UTR) of mouse or human EphrinB1, RGS3, KIF20A or SEPT7.

The sequence of mouse Kif20a cDNA and 3′ UTR is shown below. The underlined and italicized sequences are additional candidate shRNA sequences (the sense or passenger strand) predicted by web-based shRNA design tool. The underlined and bold sequences are validated candidate shRNA sequences (the sense or passenger strand) that could inhibit KIF20A expression in the mouse cells and these shRNAs are used in the study of KIF20A function in mouse brain development.

```
Sequence of mouse Kif20a cDNA starting with start codon ATG
and ending with stop codon TAA (SEQ ID NO: 4):
ATGTCTCACCGGATCCTTTCTCCGCCAGCGGGCTTACTCTCTGATGAGGATGTTGTA
GACTCTCCCATATTAGAATCCACAGCTGCAGATCTGAGATCTGTTGTCCGAAAGGACC
TGTTGTCAGACTGCTCTGTCATCTCTGCCTCTCTGGAGGACAAGCAGGCTCTACTGG
AGGACACCTCAGAGAAGGTGAAAGTTTACCTTCGAATTCGACCATTCTTGACTTCAGA
GTTGGACCGACAGGAAGATCAGGGCTGCGTCTGCATTGAGAATACAGAAACCCTTGT
GCTGCAGGCACCCAAAGACTCCTTTGCCTTGAAGAGTAATGAGAGGGGAGTT [symbol] [symbol]
      ACCTTTTCCCAGATATTTGGGCCAGAAGTGGGACAGGTGGC
CTTCTTCAATCTGACCATGAAGGAGAT [symbol] GCAGAACTG
GCTCATCTACACCTATGGAGTCACCAACTCGGGGAAAACGTACACCATTCAAGGTACT
AGTAAGGATGCGGGGATCCTGCCCCAGTCCCTGGCTCTCATCTTTAATAGTCTCCAG
GGCCAGCTTCATCCGACACCTGACCTGAA [symbol] AATCT [symbol] [symbol]
      ACAGGAGGAAATGAA [symbol] A
GGCCTCCAAGAGGAGGAGCTGTCCACCTCCGTGAAGAAACGTGTCCACACTGAAAGT
CGGATAGGTGCCAGCAACAGCTTTGACAGTGGCGTTGCTGGGCTGTCTTCCACCAGT
CAGTTCACCAGCAGCAGCCAGCTAGATGAAACAAGTCAATTATGGGCACAACCAGAC
ACTGTCCCAGTAAGTGTTCCAGCAGACATTCGCTTCTCTGTCTGGATCTCCTTCTTTG
AGATCTACAATGAATTGCTTTATGACCTGTTAGAACCACCTAGCCATCAGCATAAGAG
ACAGACACTTCGGCTGTGTGAGGATCAGAACGGCAATCCTTACGTGAAAGATCTCAA
TTGGATTCATGTTCGTGATGTTGAGGAAGCCTGGAAACTATTGAAAGTGGGTCGCAA
GAACCAGAGCTTTGCCAGCACCCACATGAACCAGCAATCCAGCCGCAGTCACAGCAT
CTTCTCAATCAGAATCTTGCACCTTCAGGGAGAAG [symbol] GT
GAACTGTCACTCTGTGACCTGGCTGGCTCTGAGCGCTGCAAACATCAAAAAAGTGGT
GAGCGGCTAAAGGAGGCAGGGAACATTAACACTTCTCTGCACACCCTGGGCCGCTG
TATTGCTGCCCTGCGACAGAATCAGCAGAACCGGTCAAAGCAGAACCTGATTCCTTT
CCGTGACAGCAAGTTGACTCGTGTGTTCCAAGGCTTCTTCACAGGTCGAGGTCGTTC
CTGTATGATTGTCAATGTGAATCCCTGTGCATCTACATATGACGAGACTCTTCATGCA
GCCAAATTCTCAGCCCTAGCCAGCCAGCTTGTGCACGCCCCACCTGTGCATCTGGGA
ATCCCATCCCTGCATTCATTCATCAAGAAGCACAGTCCTCAGGTTGGCCCTGGCTTAG
AGAAAGAGGACAAGGCCGACTCAGACCTTGAGGACAGTCCTGAAGACGAAGCTGAC
GTCTCCGTGTATGGCAAGGAGGAACTACTCCAGGTGGTGGAAGCCATGAAAGCGCTA
CTTTTAAAAGAACGACAAGAAAAGCTGCAGTTGGAGATACAGCTCCGAGAGGAAATTT [symbol]
      TGCAACAGCGGGAGCAGTGGTGCAGTGAGCGTTTG
GACAACCAAAAGGAACTGATGGA [symbol] GAAGATCCTGAAG
GAATCACTGACAACTTTCTACCAAGAGCAGATCCAGGAGCGGGATGAAAAAATTGAA
GAGCTAGAAACTCTGTTGCAGGAAGCCAAACAGCAGCCAGCGGCACAACAGTCAGG
GGGGCTGTCCCTCCTCCGGCGGTCACAGAGGCTGGCAGCGTCTGCCTCCACTCAGC
AGTTCCAGGAAGTTAAAGCTGAACTAGAGCAATGCAAAACAGAGTTAAGCTCTACCAC
TGCAGAGCTGCACAAGTATCAGCAAGTGTTAAAACCGCCACCCCCAGCCAAGCCTTT
CACCATTGATGTGGATAAGAAGTTAGAGGAGGGCCAGAAGAATATAAGACTGCTACG
GACAGAGCTCCAGAAACTAGGACAGTCTCTGCAGTCAGCAGAAAGAGCCTGTTGCCA
CAGCACCGGAGCAGGAAAACTTCGTCAAGCATTAACCAACTGTGATGACATCTTAATC
AAGCAGAACCAGACCC [symbol] TGGTGCTAGTGAAACTGGA
CCTTCAGAAAAAGGCAGCCTGCATTGCTGAGCAGTATCACACTGTGCTAAAGCTCCA
AGGCCAAGCCTCTGCCAAAAAGCGGCTGGGAGCAAACCAGGAAAACCAGCAACCAA
ATCATCAACCCCCAGGAAAGAAACCATTCCTGCGAAACTTACTTCCCCGAACACCTAC
CTGCCAAAGCTCAACAGACAGCAGCCCTTATGCGCGGATCTTGCGCTCACGGCACTC
TCCTTTACTCAAATCTCCCTTTGGCAAAAAATACTAA
Sequence of mouse Kif20a 3′ UTR (SEQ ID NO: 5):
AGTTGTGG [symbol] GCCCTGAGGTTGGTCAGCTGCTTAGCTG
ATAAGTCTCTTTTAAGCTTCAGCATACACCCAGAACTGTATTCAGAATTATTGTATTATA
ATCACCTATGTAATCTTGTTTTTTTTTTCATGCACAAATATTATATGGAATAAAGACATT
GTTTATGCTTTTATTTGGATTCCAAATTTAGAAAAATATAAAAAGGGGGACAGAAAATT
GATATAAATACCATCTGGCCTGTTAGACCCTTGGACAGGAACAGCAGTGGGAACCAG
CCTAATGCACACTAGGAAATGAGTACAATTTTAAATGATACCCAAGAGGCTTGGGTAG
CTTTGTCTGGCCAGGAAGTTGCTATGTCAAAACACCCACCTGCCTGTCTCCAAATGG
GATTAAAGACATGCACCACCACATGGCACCCAGTTAGTGTTCCTTCCATTAAATGGGG
AGTAAAATGTTCTGAGTACAAGAGAAGGAAGGCTAGCTCCTAACCACAGAATGATCTA
TAGCACCTACTGCACAAGCTCATTGTAGCAGACATCAAGTGGCTCTGAAACCTAGAAA
AACACAGTGAATTCTTCAACCTTGTAGATAAGGTTGAAAACCAAATTGGAGACTTTAAA
ATCCGAAGTAAAAAAGCTTCC
```

The sequence of human Kif20a cDNA is shown below. Underlined and italicized sequences are additional candidate shRNA sequences (the sense or passenger strand) predicted by web-based shRNA design tool. Bold and underlined sequences were validated candidate shRNA sequences (the sense or passenger strand) that could inhibit KIF20A expression in the human cells and these shRNAs were used in the study of KIF20A function in human medulloblastoma and glioblastoma cells. Examples of full sequence designs of DNA plasmid-based human Kif20a shRNAs including passenger strand, loop, and guide strand are shown in the working examples, materials and methods, below (SEQ ID NO: 6).

```
ATGTCGCAAGGGATCCTTTCTCCGCCAGCGGGCTTGCTGTCCGATGACG
ATGTCGTAGTTTCTCCCATGTTTGAGTCCACAGCTGCAGATTTGGGGTCTGTGGTACG
CAAGAACCTGCTATCAGACTGCTCTGTCGTCTCTACCTCCCTAGAGGACAAGCAGCA
GGTTCCATCTGAGGAC [symbol] TACTTGAGGGTTAGGCCCT
TGTTACCTTCAGAGTTGGAACGACAGGAAGATCAGGGTTGTGTCCGTATTGAGAATGT
GGAGACCCTTGTTCTACAAGCACCCAAGGACTCTTTTGCCCTGAAGAGCAATGAACG
GGGAATTGGCCAAGCCACACACAGGTTCACCTTTTCCCAGATCTTTGGGCCAGAAGT
GGGACAG [symbol] GTGAAGGAGATGGTAAAGGATGTACTCAA
AGGGCAGAACTGGCTCATCTATACATATGGAGTCACTAACTCAGGGAAAACCCACAC
GATTCAAGGTACCATCAAGGATGGAGGGATTCTCCCCCGGTCCCTGGCGCTGATCTT
CAATAGCCTCCAAGGCCAACTTCATCCAACACCTGATCTGAAGCCCTTGCTCTCCAAT
GAGGTAATCTGGCTAGACAGCAAGCAGATCCGACAGGAGGAAATGAAGAAGCTGTCC
CTGCTAAATGGAGGCCTCCAAGAGGAGGAGCTGTCCACTTCCTTGAA [symbol] [symbol]
      TCGGATAGGTACCAGCACCAGCTTCGACAGTGGCATTGCTGGGCT
CTCTTCTATCAGTCAGTGTACCAGCAGTAGCCAGCTGGATGAAACAAGTCATCGATG
GGCACAGCCAGACACTGCCCCACTACCTGTCCCGGCAAACATTCGCTTCTCCATCT [symbol] [symbol]
      ACAACGAACTGCTTTATGACCTATTAGAACCGCCTAGC
CAACAGCGCAAGAGGCAGACTTTGCGGCTATGCGAGGATCAAAATGGCAATCCCTAT
GTGAAAGATCTCAACTGGATTCATGTGCAAGATGCTGAGGAGGCCTGGAAGCTCCTA
AAAGTGGGTCGTAAGAACCAGAGCTTTGCCAGCACCCACCTCAACCAGAACTCCAGC
CGCAGTCACAGCATCTTCTCAATCAGGATCCTACACCTTCAGGGGGAAGGAGATATA
GTCCCCAAGATCAGCGAGCTGTCACTCTGTGATCTGGCTGGCTCAGAGCGCTGCAAA
GATCAGAAGAGTGGTGAACGGTTGAAGGAAGCAGGAAACATTAACACCTCTCTACAC
ACCCTGGGCCGCTGTATTGCTGCCCTTCGTCAAAACCAGCAGAACCGGTCAAAGCAG
AACCTGGTTCCCTTCCGTGACAGCAAGTTGACTCGAGTGTTCCAAGGTTTCTTCACAG
GCCGAGGCCGTTCCTGCATGATTGTCAATGTGAATCCCTGT [symbol] [symbol]
      CTTCATGTGGCCAAGTTCTCAGCCATTGCTAGCCAGCTTGTGCATGCCCCACCT
ATGCAACTGGGATTCCCATCCCTGCACTCGTTCATCAAGGAACATAGTCTTCAGGTAT
CCCCCAGCTTAGAGAAAGGGGCTAAGGCAGACACAGGCCTTGATGATGATATTGAAA
ATGAAGCTGACATCTCCATGTATGGCAAAGAGGAGCTCCTACAAGTTGTGGAAGCCA
TGAAGACACTGCTTTTGAAGGAACGACAGGAAAAGCTACAGCT [symbol] [symbol]
      AATTTGCAATGAGATGGTAGAACAGATGCAACAGCG [symbol] [symbol]
      TTTGGACACCCAAAAGGAACTATTGGAGGAAATGTATGAAGAAAAACTAAA
TATCCTCAAGGAGTCACTGACAAGTTTTTACCAAGAAGAGATTCAGGAGCGGGATGAA
AAGATTGAAGAGCTAGAAGCTCTCTTGCAGGAAGCCAGACAACAGTCAGTGGCCCAT
CAGCAATCAGGGTCTGAATTGGCCCTACGGCGGTCACAAAGGTTGGCAGCTTCTGCC
TCCACCCA [symbol] TAAATTACA [symbol] [symbol]
      CTCTACCACTGAAGAGTTGCATAAGTATCAGAAAATGTTAGAACCACCACCCTCAG
CCAAGCCCTTCACCATTGATGTGGACAAGAAGTTAGAAGAGGGCCAGAAGAATATAA
      G [symbol] GAAACTTGGTGAGTCTCTCCAATCAGCAGAGAGAG
CTTGTTGCCACAGCACTGGGGCAGGAAAACTTCGTCAAGCCTTGACCACTTGTGATG
ACATCTTAATCAAACAGGACCAGACTCTGGCTGAACT [symbol]
GAAACTGGACCTTCGGAAGAAGGCAGCATGTATTGCTGAGCAGTATCATACTGTGTT
GAAACTCCAAGGCCAGGTTTCTGCCAAAAAGCGCCTTGGTACCAACCAGGAAAATCA
GCAACCAAACCAACAACCACCAGGGAAGAAACCATTCCTTCGAAATTTACTTCCCCGA
ACACCAACCTGCCAAAGCTCAACAGACTGCAGCCCTTATGCCCGGATCCTACGCTCA
CGGCGTTCCCCTTTACTCAAATCTGGGCCTTTTGGCAAAAAGTACTAA
```

The sequence of mouse EphrinB1 CDS and 3′ UTR of the cDNA is shown below. The underlined and italicized sequences are additional candidate shRNA sequences predicted by web-based shRNA design tool. The bold and underlined sequences were validated candidate shRNA sequences that could inhibit the expression of EphrinB1 in the mouse cells and these shRNAs were used in the study of the function of these proteins in mouse brain development.

```
Sequence of mouse EphrinB1 cDNA starting with start codon ATG and
ending with stop codon TGA (SEQ ID NO: 7):
ATGGCCCGGCCTG [symbol] [symbol] TCGTGCT
GACGCTGTGCCGGCTTGCCACGCCGTTGGCCAAGAACCTGGAGCCCGTGTCCT [symbol] [symbol]
      TCCTAAGTGGGAAGGGCTT [symbol] [symbol]
      AGACAAGCTGGACATCATCTGCCCCCGAGCAGAAGCAGGGC [symbol] [symbol]
      TGTACCTGGTGCGGCCAGAGCAGGCGGCTGCTTGCAGCA [symbol] [symbol]
      TACTGGTCACTTGCAACAAGCCACACCAGGAAATCCGCTTC [symbol] [symbol]
      AGCCCCAACTACATGGGCCTGGAATTCAAAAAGTACCACGATTA
CTACATTACATCAACGTCCAATGGGAGCTTGGAGGGACTGGAGAACCGGGAGGGAG
GTGTGTGTCGCACCC [symbol] AGGTTGGGCAAGATCCAAATG
CTGTGACACCCGAGCAGTTGACTACCAGCCGGCCAAGCAAGGAGTCAGACAACACT
GTCAAGACAGCCACACAGGCTCCTGGTCGGGGATCCCAGGGTGACTCTGACGGCAA
GCATGAGACTGTGAACCAGGAAGAGAAGAGTGGCCCAGGTGCAGGTGGCGGTGGCA
GCGGGGACTCTGACAGCTTCTTCAACTCCAAGGTAGCATTGTTCGCAGCCGTCGGCG
CCGGCTGTGTCATCTTCCTGCTCATCATCATCTTCTTGACAGTCCTACTACTCAAGCT
CCGCAAGCGCCATCGCAAGCATACACAGCAGCGGGCGGCTGCCCTCTCGCTCAGTA
CCCTAGCCAGCCCCAAAGGGGGTAGTGGTACAGCGGGCACCGAGCCCAGCGACATC
ATCATCCCCTTACGGACTACAGAGAACAACTACTGCCCCCACTATGAGAAGGTGAGT
GGGGACTACGGGCATCCTGTCTACATCGTCCAGGAGATGCCCCCTCAGA [symbol] [symbol]
      AGGTTTGA
Sequence of mouse EphrinB1 3′ UTR (SEQ ID NO: 8):
AGGCCCAGCATGGCCTCAGGCCCCACAGGACACATGGCCTGGATCGGCCGCTCTCT
TCTCCCCCACACCCCTGCCCTTGCCAGCTGTGCCCACCTTTGTATTTAGTTTTGTAGT
TTCTTGGCTTTTATAATCCCCTTTTTCTTGCCCCCTGGGCTTCGGAGGGGGGTGCTTG
TGCCCCCAATCCCCATGCTCTTGTGCCTTCCCCCTCTGGCCAGGCCTCTGGGCTCCG
TGGGGGTGCCCTTTCTAGGAGGGCAGGGTTGGACACTGACGGACTGCAGGCAGGGA
GGTAGCCCCCTGGCCCTGCCCCCTCCTCATCCCTTCACCCATCCCCAG [symbol] [symbol]
      ACTGTTTTTAATGCTTTTGTGTTGATTTTTTTTAAAAGCTGTCAACT
CATTTTCATCTGTTTTTTTTTTTTTGAGAAAAAAAAATGGGAAAATGTACAAGGCAGCC
CCTTCCCAGGCTTTCTGAGCCTGGCCTGACCCAGTCTAAGAGGGCCTGGGGCAGGA
TGTGGCCAGCCAGGAAACATAGGATGGCATTTCTTTTATAGACTCCTTAGTAGTTCAG
GGGAGTGGGGGA [symbol] GCCCACCGAGGAGAGAAGAGCG
CCACCCTCCTTTTTGGACCCTCATGAGACAATGCTGTTCCTAGCAGTGG [symbol] [symbol]
      ACCAAAAAAAAAAAAAAAG
```

The sequence of mouse RGS3 CDS of the cDNA is shown below. The underlined and italicized sequences are additional candidate shRNA sequences predicted by web-based shRNA design tool. The bold and underlined sequences were validated candidate shRNA sequences that could inhibit the expression of RGS3 in the mouse cells and these shRNAs were used in the study of the function of these proteins in mouse brain development (SEQ ID NO: 9).

```
ATGAACC [symbol] TGTGTTCAGAACGCCGCTACC
GGCAGATCACCATCCGGAGGGGCAAAGACGGCTTTGGCTTCACCATCTGCTGTGACT
CTCCGGTCCGAGTCCAGGCTGTGGATTCTGGGGGCCCGGCAGAGAGGGGGGACT
GCAGCAGCTGGACACAGTGCTACAACTGAATGAGAGACCCGTGGAGCACTGGAAAT
GTGTGGAGCTGGCACATGAGATCCGGAGCTGTCCTAGCGAGATCATCCTGCTCGTGT
GGCGTGTGGTCCCCCAGATCAAGCCGGGGCCAGATGGCGGAGTCTTGCGGGGGGC
CTCCTGCAAGTCCACACATGACCTCCTGTCACCCCCTAACAAGAGGGAGAAGAACTG
TACTCATGGGGCCCCAGTTCGTCCTGAGCAGCGCCACAGCTGCCACCTGGTGTGTG
ACAGCTCTGATGGTCTACTGCTTGGTGGCTGGGAGCGCTACACTGAGGTGGGCAAG
CGCAGTGGCCAGCACACCCTGCCTGCACTGTCCCGGACCACCACCCCTACTGACCC
CAACTACATCATCCTGGCCCCACTGAATCCTGGAAGCCAGTTGCTGCGGCCTGTGTA
CCAGGAGGATACAATCCCTGAAGAACCGGGGACTACTACTAAAGGGAAATCGTACAC
CGGCCTGGGCAAGAAGTCTCGGCTCATGAAGACAGTGCAGACCATGAAGGGCCACA
GTAACTACCAAGACTGCTCAGCCCTGAGACCGCACATCCCGCATTCCAGTTACGGCA
CCTATGTCACCCTGGCCCCTAAAGTCCTGGTGTTCCCTGTCTTTGTGCAGCCCCTAG
ATCTCTGTAACCCTGCCCGGACTCTCCTGCTGTCGGAGGAGCTGCTGCTGTATGAGG
GGAGGAACAAGACTTCCCAGGTGACACTGTTTGCCTACTCGGACCTGCTGCTGTTCA
CTAAGGAGGAGGAGCCAGGCCGCTGCGACGTCCTGAGAAATCCCCTCTACCTCCAG
AGCGTGAAGCTACAGGAGGGCTCTTCAGAAGACTTGAAATTCTGTGTGCTGTACCTG
GCAGAGAAGGCAGAGTGCTTATTCACTTTGGAGGCACACTCGCAGGAGCAGAAGAA
GAGAGTGTGCTGGTGCCTGTCGGAGAACATCGCCAAGCAGCAACAGCTGGCCGCAC
CACCTAC [symbol] AGAGGCAGATGAGAAGGAGATGCCCCTG
GTCGAGGGGAAGGGGCCAGGTGCTGAGGAACCCGCACCCAGCAAAAATCCCTCTCC
TGGCCAGGAGCTTCCTCCAGGACAAGACCTCCCTCCCAGCAAAGACCCCTCTCCCAG
CCAGGAGCTTCCTGCAGGACAAGATCTCCCACCCAGGAAGGACTCCCCAGGCCAAG
AAGCTGCTCCTGGCCCAGAATCACCATCCAGTGAAGACATAGCAACCTGCCCTAAGC
CCCCTCAAA [symbol] AGGACTCCCCACCAGGCCAGGGATCCT
CCCCGACCACAGAACTCCCATCTTGCCAGGGCCTTCCT [symbol] [symbol]
      CAGGACCCTCTGCTCAGTCAAGAGCCCCCTGTTATCCCAGAATCCTCTGCCTCCG
TCCAGAAACGCCTACCCTCTCAGGAGTCACCCTCCAGCCTGGGCTCCCTGCCAGAGA
AGGACCTTGCTGAGCAGACCATCAGCTCCGGGGAGCCACCAGTCGCCACTGGTGCT
GTACT [symbol] TGTGATCCCCGAGGT [symbol] [symbol]
      CCAACTGGATGGGGCCCACGGAGGCAGCTCTGGCGAGGACGAGGATGCAG
AAGAGGGAGAGGAGGGGGGAGAAGGGGAGGAAGATGAGGAGGACGACACCAGCGA
CGACAACTACGGGGATCGTAGTGAGGCCAAGCGCAGCAGCCTGATTGAGACTGGCC
AAGGCGCCGAGGGCGGCTTCTCGTTGCGTGTGCAGAACTCGCTGCGGCGCCGGAC
GCACAGCGAGGGCAGCCTGCTGCAGGAGTCCCGGGGGCCCTGCTTTGCCTCTGACA
CCACCTTACACTGCTCTGATGGCGAGGGCGCCACATCCACCTGGGCTATCCCTTCAC
CCCGCACCCTCAAAAAAGAACTGGGTCGTAATGGAGGCTCCATGCACCACCTTTCCC
TGTTCTTCACGGGACACAGGAAGATGAGT [symbol] GAT [symbol] [symbol]
      AGCAAAAACA [symbol] CTGGCCA
TCTTCAGGCGGCGGAACGAATCTCCCGGGGCTCAGCCAGCCAGCAAGACAGACAAG
ACAACCAAGTCCTTCAAGCCTACCTCGGAGGAAGCCCTCAAGTGGAGCGAGTCCCTG
GAAAAGTTGCTGCTTCATAAATATGGCTTAGAAGTGTTCCAGGCCTTCCTTCGCACCG
AGTTCAGTGAGGAGAACCTGGAGTTTTGGCTCGCTTGCGAGGACTTCAAGAAGGTCA
AATCACAGTCCAAGATGGCAGCCAAAGCCAAGAAGATCTTTGCTGAATTCATCGCAAT
CCAGGCTTGCAAAGAGGTAAACCTGGACTCCTACACACGAGAACACACCAAGGAGAA
TCTGCAGAGCATCACCCGAGGCTGCTTTGACTTGGCACAGAAACGTATCTTTGGGCT
CATGGAGAAGGACTCTTACCCTCGCTTCCTCCGCTCTGACCTCTACCTGGACCTCATT
AACCAGAAGAAGATGAGTCCCCCGCTCTAG
```

The sequence of mouse SEPT7 CDS and 3′ UTR of the cDNA is shown below. The underlined and italicized sequences are additional candidate shRNA sequences predicted by web-based shRNA design tool. The bold and underlined sequences were validated candidate shRNA sequences that could inhibit the expression of SEPT7 in the mouse cells and these shRNAs were used in the study of the function of these proteins in mouse brain development.

```
Sequence of mouse SEPT7 cDNA starting with start codon ATG and
ending with stop codon TAA (SEQ ID NO: 10):
ATGTCGGTCAGTGCGAGATCCGCTGCTGCCGAGGAGAGGAGCGTCAACTGCGGCAC
CATGGCTCAACCGAAGAACCTTGAGGGCTATGTGGGCTTTGCCAACCTCCCAAATCA
AGTGTACAGAAAATCCGTGAAAAGAGGATTTGAATTCACTCTTATGGTAGTAGGTGAA
TCTGGACTGGGAAAGTCGACATTAATCAACTCATTATTCCTCACAGATTTGTATTCTCC
AGAGTATCCAGGACCTTCTCATAGAATCAAAAAGACTGTACAGGTGGAGCAATCCAAA
GTTTTAATCAAAGAAGGTGGTGTTCAGTTGCTGCTGACAATAGTTGATACTCCAGGAT
TTGGAGATGCAGTGGATAATAGTAATTGCTGGCAGCCTGTTATCGACTACATTGATAG
TAAATTTGAAGATTACTTAAATGCAGAATCTCGAGTGAACAGACGTCAGATGCCTGAT
AACAGGGTGCAGTGTTGTTTATACTTCATTGCTCCTTCAGGACATGGACTTAAACCAT
TGGACATTGAGTTTATGAAACGTTTGCATGAAAAAGTGAATATCATCCCATTAATTGCC
AAAGCAGACACACTTACACCAGAGGAATGCCAACAGTTTAAAAAGCAGATAATGAAAG
AAATTCAAGAACATAAAATTAAAATATATGAATTTCCAGAAACAGATGATGAAGAAGAG
AATAAACT [symbol] CCTCTT [symbol] [symbol]
      ATCATTGAAGTTAATGGCAAAAGAGTCAGAGGAAGGCAGTATCCTTGGGGTGTGG
CTGAAGTTGAAAATGGTGAACATTGTGATTTCACAATTCTAAGAAATATGTTGATAAGA
ACACATATG [symbol] ACGTACACTATGAGAACTACAGAA
GCAGAAAGCTAGCAGCAGTGACTTACAATGGAGTGGATAACAACAAGAACAAAGGGC
AGCTTACCAAGAGCCCTCTGGCACAGATGGAAGAAGAAAGAAGGGAACATGTAGCCA
AAATGAAGAAGATGGAGATGGAGATG [symbol] AAGAAA
AAGTTCAAAAACTGAAGGACTCTGAAGCAGAGCTCCAGCGGCGCCATGAGCAAATGA
AAAAGAATTTAGAAGCACAGCACAAAGAATTAGAGGAAAAACGTCGTCAGTTTGAAGA
AGAAAAGGCAAACTGGGAAGCTCAACAGCGTATTTTAGAGCAA [symbol] [symbol]
      AGAACAAGAAGAAAGGCAAGATCTTTTAA
Sequence of mouse SEPT7 3′ UTR (SEQ ID NO: 11):
ACTCTATTGACCACCAGTTATGTATTAGTTGCCAATATGCCAGCTTG [symbol] [symbol]
      TGACCAATTTGCACCAATTTTATCCATAATGATGGATTTAACAGCA
TGACAAAAATTATTTTTTGTTGTTCTCGAAGGAGATTAAGATGC [symbol] [symbol]
      TGTACTTAGAAATTAACAGCTCTAAGTACCTTTCTACATTTTTTTTCTTTTTTTT
TTAAATTAAAAGATGTCTTCAGTTTAATGCAAGAAAACATTTTACTGTTGTACAATCATG
TTCTGGTGGTTTGATTGTTTACAGAATATTCTAAAATAAAAGGACTCTGGAAGGTTTTC
ATTGAGGATAAATTGCCATAATATGATGCAAACTATGCTTCTCTATGATAATTATAATAC
AGAGGTT [symbol] TGTATTTAGTCTAACACAGTGGACCC
TATTTTTTGACACTTCCATTGTTTAAAAGTACACATGGAAAAAACATATATGCTTACAGT
GCACCTAGAGCTTTTTATAACAGCCTTTTTTGTTTGTTTGTTTGTTTTGGATTCTTTAAA
TATATAAATATATTATTCTCATTTAGTGCCCCTGTAGCCAGAACCTCATTACTGCTTCAT
TTTTGTAATAACATTTAATTTAGATATTTTCCATATATTGGCCCTGCTAAAATAGAATAT
AGCATCTTTCATATGGTAGGAACCAACGAGGAAACTTTCCTTTAACTCCCTTTTTACAC
TTTATGGAAAGTAGCAGGAGGAGAAATGCATTTGTAGATCATTTCTAGGCAAATTGTG
AAGCTAA [symbol] CAGTCTTGTTTTACTAGAAATGGGAAT
CATGGCCTCTTGAAGAGAAAAAAGT [symbol] ATATATTGC
ATACCTGTATTTTTTTGTTTGTATTGTAAAAAAAATTCACATAATAAACAATGTTGTGAT
GTAA
```

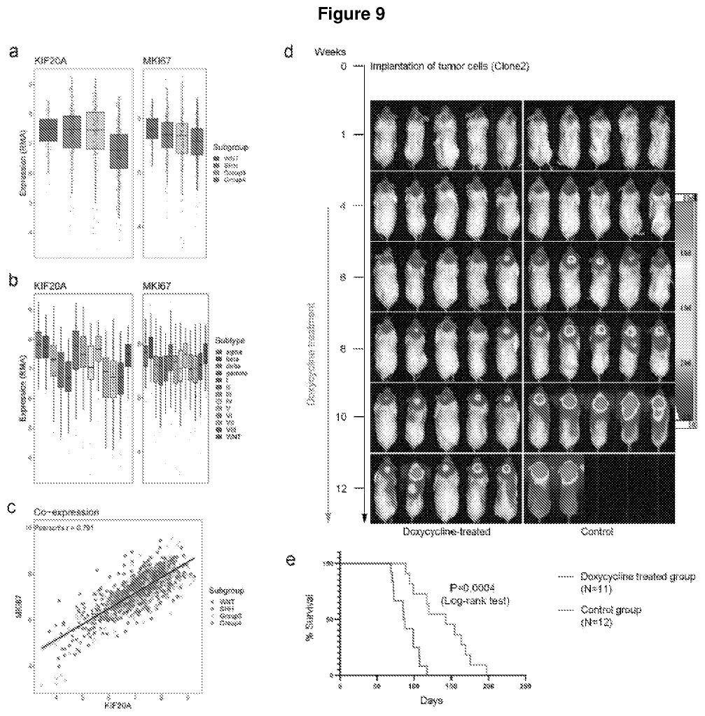

Of these potential shRNA sequences, multiple candidate sequences spanning different regions of individual cDNA were experimentally tested. The shRNAs that are validated for specific knockdown of target protein expression are used in previous functional studies in the mouse cortex. Two shRNAs specifically targeting the CDS or 3′-UTR of human KIF20A transcript were also developed. Experiments using the shRNAs for human KIF20A showed that knockdown of KIF20A in human medulloblastoma cells or human glioblastoma cells could inhibit cell proliferation and suppress brain tumor growth (FIGS. 9d-e, 10c, 11c, 12a-b).

In some embodiments, examples of shRNAs and siRNAs are disclosed in Table 1 below. All sequences shown below represent the passenger (sense) strand of shRNAs or siRNAs. The guide (anti-sense) strand sequences of shRNAs or siRNAs are reverse complement to the passenger strand sequences. Example of a DNA plasmid-based shRNA in full sequence including passenger strand, loop, and guide strand was shown in the working examples. siRNAs are double-stranded RNA oligos formed by the passenger (sense) strand and guide (anti-sense) strand.

TABLE 1
Examples of shRNAs and siRNAs

| | | SEQ | | SEQ ID |
|---|---|---|---|---|
| Target | shRNA Sequence | ID NO. | siRNA Sequence | NO. |
| Mouse | GGTCAGGCCACTCA | 12 | GGUCAGGCCACUCACAAA | 13 |
| Kif20a | CAAATTT | | UUU | |
| CDS | | | | |
| Mouse | GGTGAAGGATGTGC | 14 | GGUGAAGGAUGUGCUCA | 15 |
| Kif20a | TCAAAGG | | AAGG | |
| CDS | | | | |
| Mouse | GCCCTTATTGTCCAA | 16 | GCCCUUAUUGUCCAAUGA | 17 |
| Kif20a | TGAAGT | | AGU | |
| CDS | | | | |
| Mouse | GGCTAGACAGCAAG | 18 | GGCUAGACAGCAAGCAGA | 19 |
| Kif20a | CAGATTCG | | UUCG | |
| CDS | | | | |
| Mouse | GAAGCTGTCCTTGCT | 20 | GAAGCUGUCCUUGCUAA | 21 |
| Kif20a | AATTGG | | UUGG | |
| CDS | | | | |
| Mouse | GGGATATAGTGCCC | 22 | GGGAUAUAGUGCCCAAG | 23 |
| Kif20a | AAGATCA | | AUCA | |
| CDS | | | | |
| Mouse | GCAACGAAATGGTG | 24 | GCAACGAAAUGGUGGAAC | 25 |
| Kif20a | GAACAGA | | AGA | |
| CDS | | | | |
| Mouse | GGAACTGTACGAGG | 26 | GGAACUGUACGAGGAGA | 27 |
| Kif20a | AGAAACT | | AACU | |
| CDS | | | | |
| Mouse | TGGCTGAGCTGCAG | 28 | UGGCUGAGCUGCAGAAU | 29 |
| Kif20a | AATAACA* | | AACA | |
| CDS | | | | |
| Mouse | GGATGAAGATGCAT | 30 | GGAUGAAGAUGCAUGGU | 31 |
| Kif20a 3′ | GGTTGTT* | | UGUU | |
| UTR | | | | |
| Human | AGTATGGAGAAGGT | 32 | AGUAUGGAGAAGGUGAAA | 33 |
| Kif20A | GAAAGTA* | | GUA | |
| Human | GCATCCTTCTTCAAC | 34 | GCAUCCUUCUUCAACCUA | 35 |
| Kif20A | CTAACT | | ACU | |
| Human | GAGGAGTGTCTACAT | 36 | GAGGAGUGUCUACAUCG | 37 |
| Kif20A | CGAAAG* | | AAAG | |
| Human | GGATCTCATTCTTTG | 38 | GGAUCUCAUUCUUUGAG | 39 |
| Kif20A | AGATCT | | AUCU | |
| Human | GCATCTACCTATGAT | 40 | GCAUCUACCUAUGAUGAA | 41 |
| Kif20A | GAAACT | | ACU | |
| Human | GGAGATGCATCTCC | 42 | GGAGAUGCAUCUCCGAG | 43 |
| Kif20A | GAGATGA | | AUGA | |
| Human | GGAACAGTGGTGCA | 44 | GGAACAGUGGUGCAGUG | 45 |
| Kif20A | GTGAACA | | AACA | |
| Human | GCAGCTTCAGGAGG | 46 | GCAGCUUCAGGAGGUUA | 47 |
| Kif20A | TTAAAGC | | AAGC | |
| Human | GCAGTGCAAAGCAG | 48 | GCAGUGCAAAGCAGAGC | 49 |
| Kif20A | AGCTAAA | | UAAA | |
| Human | GCTGTTGCGGACAG | 50 | GCUGUUGCGGACAGAGC | 51 |
| Kif20A | AGCTTCA | | UUCA | |
| Human | GCAGAACAACATGGT | 52 | GCAGAACAACAUGGUGCU | 53 |
| Kif20A | GCTAGT | | AGU | |
| Mouse | GGCAGCGTTGGCTC | 54 | GGCAGCGUUGGCUCAGC | 55 |
| EphrinB1 | AGCAAGT | | AAGU | |
| CDS | | | | |
| Mouse | GCAAGTGGCTTGTG | 56 | GCAAGUGGCUUGUGGCU | 57 |
| EphrinB1 | GCTATGG | | AUGG | |
| CDS | | | | |
| Mouse | GGAGCTCTCTTAACC | 58 | GGAGCUCUCUUAACCCUA | 59 |
| EphrinB1 | CTAAGT | | AGU | |
| CDS | | | | |
| Mouse | GGTGATCTACCCGAA | 60 | GGUGAUCUACCCGAAGA | 61 |
| EphrinB1 | GATTGG | | UUGG | |
| CDS | | | | |
| Mouse | GGCCCTACGAGTAC | 62 | GGCCCUACGAGUACUACA | 63 |
| EphrinB1 | TACAAGC | | AGC | |
| CDS | | | | |
| Mouse | GCACTGTGCTTGATC | 64 | GCACUGUGCUUGAUCCC | 65 |
| EphrinB1 | CCAATG | | AAUG | |
| CDS | | | | |
| Mouse | ACCATCAAGTTCCAA | 66 | ACCAUCAAGUUCCAAGAG | 67 |
| EphrinB1 | GAGTTC* | | UUC | |
| CDS | | | | |
| Mouse | GCACTATGAAGATCG | 68 | GCACUAUGAAGAUCGUUA | 69 |
| EphrinB1 | TTATGA | | UGA | |
| CDS | | | | |
| Mouse | GCCCGGCGAACATC | 70 | GCCCGGCGAACAUCUAC | 71 |
| EphrinB1 | TACTACA | | UACA | |
| CDS | | | | |
| Mouse | GACTGCTCGTCCACT | 72 | GACUGCUCGUCCACUAU | 73 |
| EphrinB1 | ATCATC* | | CAUC | |
| 3′ UTR | | | | |
| Mouse | GGGTAGGCCAGGGC | 74 | GGGUAGGCCAGGGCUAU | 75 |
| EphrinB1 | TATTTCT | | UUCU | |
| 3′ UTR | | | | |
| Mouse | GGTAGTGACTACCAC | 76 | GGUAGUGACUACCACCC | 77 |
| EphrinB1 | CCTTCT | | UUCU | |
| 3′ UTR | | | | |
| Mouse | GCTTCAATGGGCTCT | 78 | GCUUCAAUGGGCUCUGC | 79 |
| PDZ- | GCAAAG | | AAAG | |
| RGS3 | | | | |
| CDS | | | | |
| Mouse | AGAGAGGAAGATGTT | 80 | AGAGAGGAAGAUGUUUG | 81 |
| PDZ- | TGAGAC* | | AGAC | |
| RGS3 | | | | |
| CDS | | | | |
| Mouse | GCCCAGAAACCTCAA | 82 | GCCCAGAAACCUCAACAA | 83 |
| PDZ- | CAAGCA | | GCA | |
| RGS3 | | | | |
| CDS | | | | |
| Mouse | GCTGGTCAAGAATCT | 84 | GCUGGUCAAGAAUCUACU | 85 |
| PDZ- | ACTAGC | | AGC | |
| RGS3 | | | | |
| CDS | | | | |
| Mouse | GCCAGCCTCTAGAC | 86 | GCCAGCCUCUAGACCUAA | 87 |
| PDZ- | CTAACTT | | CUU | |
| RGS3 | | | | |
| CDS | | | | |
| Mouse | GCGTCTGGATAATGC | 88 | GCGUCUGGAUAAUGCCU | 89 |
| PDZ- | CTACAG | | ACAG | |
| RGS3 | | | | |
| CDS | | | | |
| Mouse | GGGACTGACCTCAC | 90 | GGGACUGACCUCACAGAA | 91 |
| PDZ- | AGAATGT | | UGU | |
| RGS3 | | | | |
| CDS | | | | |
| Mouse | GAAGCTTCCCGGAA | 92 | GAAGCUUCCCGGAAGAG | 93 |
| PDZ- | GAGAAAG | | AAAG | |
| RGS3 | | | | |
| CDS | | | | |
| Mouse | GCCAAGGACATGAA | 94 | GCCAAGGACAUGAAGAAU | 95 |
| PDZ- | GAATAAG | | AAG | |
| RGS3 | | | | |
| CDS | | | | |
| Mouse | GGTTAAGAAGATAAA | 96 | GGUUAAGAAGAUAAAGGA | 97 |
| SEPT7 | GGACCGTTTA | | CCGUUUA | |
| CDS | | | | |
| Mouse | GCTGTGGTAGGTAG | 98 | GCUGUGGUAGGUAGUAA | 99 |
| SEPT7 | TAATACT* | | UACU | |
| CDS | | | | |
| Mouse | CAGGACTTGAAAGAT | 100 | CAGGACUUGAAAGAUGUU | 101 |
| SEPT7 | GTTACCAATA | | ACCAAUA | |
| CDS | | | | |
| Mouse | GAACAGGTGTTTGAG | 102 | GAACAGGUGUUUGAGAU | 103 |
| SEPT7 | ATGAAGGTCA | | GAAGGUCA | |
| CDS | | | | |
| Mouse | CAGAACTCTTCAAGA | 104 | CAGAACUCUUCAAGAACC | 105 |
| SEPT7 | ACCTTGGAAA | | UUGGAAA | |
| CDS | | | | |
| Mouse | GACATCAGTGTTTGT | 106 | GACAUCAGUGUUUGUUG | 107 |
| SEPT7 | TGGATCCGTT | | GAUCCGUU | |
| 3′ UTR | | | | |
| Mouse | CTTGAATTGTCTAGG | 108 | CUUGAAUUGUCUAGGAUA | 109 |
| SEPT7 | ATATTG* | | UUG | |
| 3′ UTR | | | | |
| Mouse | CCATTCGATGCAGCC | 110 | CCAUUCGAUGCAGCCUAU | 111 |
| SEPT7 | TATACAATAA | | ACAAUAA | |
| 3′ UTR | | | | |
| Mouse | CGACCAGCCTGTTTC | 112 | CGACCAGCCUGUUUCUU | 113 |
| SEPT7 | TTCCTATACT | | CCUAUACU | |
| 3′ UTR | | | | |
| Mouse | CACCATTCTGCATTT | 114 | CACCAUUCUGCAUUUAGC | 115 |
| SEPT7 | AGCTGTATCT | | UGUAUCU | |
| 3′ UTR | | | | |

*Denotes the shRNAs that have been validated.

In a previously published study, the binding domain of KIF20A for interacting with RGS3 was mapped to between amino acid residues 625 and 818 within the mouse KIF20A sequence⁹. This peptide of KIF20A (KIF20A₆₂₅₋₈₁₈) also interacts with SEPT7¹⁰. In addition, mouse KIF20A₆₂₅₋₈₁₈ was able to block interaction between KIF20A and RGS3 and cause neuronal differentiation when expressed in normal neural stem/progenitor cells, suggesting that the mouse KIF20A₆₂₅₋₈₁₈ peptide or human KIF20A₆₂₆₋₈₂₀ and fragments thereof could function as a competitive inhibitor of the KIF20A-RGS3 binding. Accordingly, in some embodiments, the peptide inhibitor disclosed herein has an amino acid sequence of at least 75%, at least 80%, at least 85%, at least 90%, at least 95%, at least 98%, at least 99%, or 100% identical to the amino acid sequence of residues 625-818 of mouse KIF20A or residues 626-820 of human KIF20A. In some embodiments, the peptide inhibitor comprises an amino acid sequence of at least 75%, at least 80%, at least 85%, at least 90%, at least 95%, at least 98%, at least 99%, or 100% identical to human KIF20A fragment having an amino acid sequence below (SEQ ID NO: 116):

LNILKESLTSFYQEEIQERDEKIEELEALLQEARQQSVAHQQSGSELAL
RRSQRLAASASTQQLQEVKAKLQQCKAELNSTTEELHKYQKMLEPPPSA
KPFTIDVDKKLEEGQKNIRLLRTELQKLGESLQSAERACCHSTGAGKLR
QALTTCDDILIKQDQTLAELQNNMVLVKLDLRKKAACIAEQYHTVLKL

In certain embodiments, the peptide inhibitor is a small peptide corresponding to the C-terminus of the conserved EphrinB proteins, which blocks the binding between EphrinB and RGS3. In certain embodiments, the peptide inhibitor is a small peptide comprising 33, 32, 31, 30, 29, 28, 27, 26, 25, 24, 23, 22, 21, 20, 19, 18, 17, 16, 15, 14, 13, 12, 11, 10, 9, 8, 7, or 6 amino acid residues or less of the C-terminus of the conserved EphrinB proteins. In certain embodiments, the peptide inhibitor comprises an amino acid sequence at least 75%, at least 80%, at least 85%, at least 90%, at least 95%, at least 98%, at least 99%, or 100% identical to the amino acid sequence of a small peptide comprising 33, 32, 31, 30, 29, 28, 27, 26, 25, 24, 23, 22, 21, 20, 19, 18, 17, 16, 15, 14, 13, 12, 11, 10, 9, 8, 7, or 6 amino acid residues or less of the C-terminus of the conserved EphrinB proteins.

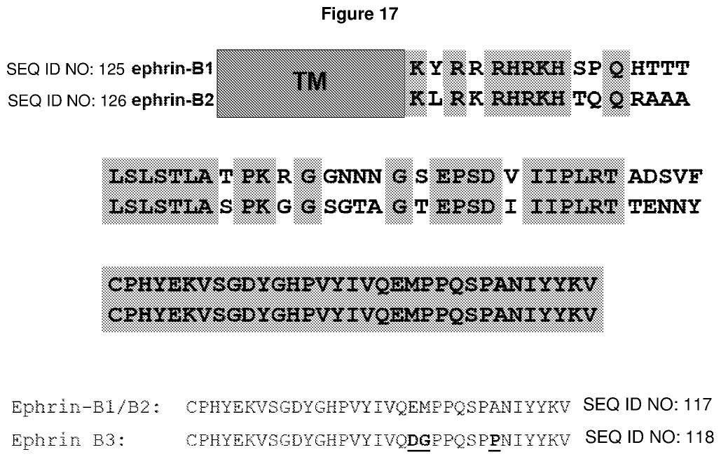

FIG. 17 shows an example of the alignment of the EphrinB1 and B2 proteins. The conserved C-terminal tail sequence (33 amino acids) between EphrinB1 and B2 is 100% identify between mouse and human. For EphrinB3, this sequence is 91% identical to Ephrin-B1/B2, as depicted in the sequence alignment below:

Ephrin-B1/B2:
(SEQ ID NO: 117)
CPHYEKVSGDYGHPVYIVQEMPPQSPANIYYKV
Ephrin B3:
(SEQ ID NO: 118)
CPHYEKVSGDYGHPVYIVQDGPPQSPPNIYYKV

In some embodiments, the small peptide comprises an amino acid sequence at least 75%, at least 80%, at least 85%, at least 90%, at least 95%, at least 98%, at least 99%, or 100% identical to the amino acid sequence of SPANIYYKV (SEQ ID NO: 1). In some embodiments, the small peptide comprises an amino acid sequence at least 75%, at least 80%, at least 85%, at least 90%, at least 95%, at least 98%, at least 99%, or 100% identical to the amino acid sequence of ANIYYKV (SEQ ID NO: 2). In some embodiments, the small peptide comprises an amino acid sequence at least 75%, at least 80%, at least 85%, at least 90%, at least 95%, at least 98%, at least 99%, or 100% identical to the amino acid sequence of NIYYKV (SEQ ID NO: 3).

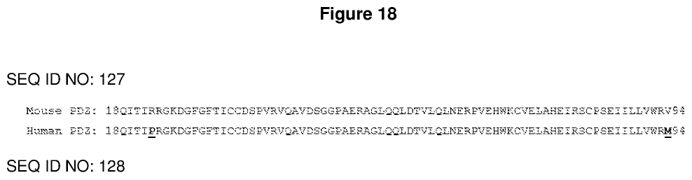

In certain embodiments, the peptide inhibitor is a peptide comprising an amino acid sequence at least 75%, at least 80%, at least 85%, at least 90%, at least 95%, at least 98%, at least 99%, or 100% identical to the amino acid sequence of the PDZ domain of mouse or human RGS3 isoform 1 (PDZ-RGS3) residues 18-94. The amino acid sequence of human PDZ domain of RGS3 is shown below (SEQ ID NO: 119): QITIPRGKDGFGFTICCDSPVRVQAVDSGGPAERAGLQQLDTVLQLNERPVEHWKCVEL AHEIRSCPSEIILLVWRM. FIG. 18 shows the alignment of mouse PDZ domain and human PDZ domain.

During mammalian brain development, extensive expansion occurs in neural progenitor cells (NPCs), but in contrast to the proliferating cells in a growing tumor, NPCs can produce correct numbers of diverse cell types without running a risk of excessive or dysregulated proliferation. NPCs achieve this feat through coordinated actions of growth stimulating and suppressing factors leading to symmetric or asymmetric cell divisions which endow a proliferative or differentiative fate to daughter cells, so that an overall balance between proliferation and differentiation is ensured throughout brain morphogenesis. Disruption of the balance between symmetric and asymmetric cell divisions can cause imbalance between proliferation and differentiation and may lead to tumorigenesis. Genetic studies in the Drosophila nervous system presented early evidence linking defects in cell division mode to tumorigenesis. In the Drosophila neuroblasts, the fly NPCs, mutations of multiple regulators of asymmetric cell division have been linked to hyperproliferation of neuroblasts in the embryos¹⁻⁶ and tumor-like growth in recipient flies when mutant cells were transplanted¹. These observations indicate that mis-regulation of the fate of an NPC's daughter cells could lead to cancer^{7, 8}. These observations also suggest that the fate specification process of daughter cells might serve as a point of intervention for developing cancer therapy. However, key regulators that control symmetric proliferative vs. asymmetric differentiative divisions in the mammalian NPCs have remained largely elusive, and hence the idea of targeting regulators of cell division mode for therapeutic intervention has not been directly tested in the mammalian systems.

KIF20A, a mitotic kinesin previously implicated in cytokinesis regulation in cancer cells, plays a crucial role in balancing symmetric versus asymmetric NPC divisions during cerebral cortical development⁹. The working examples show that inactivation of KIF20A through either shRNA knockdown or genetic knockout caused the affected cortical NPCs to switch from proliferative to differentiative mode of divisions. KIF20A exerts its function in the process of daughter cell fate specification during NPC divisions in coordination with RGS3⁹ and SEPT7¹⁰. As tumor-initiating (stem/progenitor-like) cells share many regulatory mechanisms of cell proliferation with normal neural stem/progenitor cells¹¹⁻KIF20A can be crucial for the control of proliferation versus differentiation of tumor-initiating cells.

In human cancers, KIF20A shows low mutation rate based on available TCGA database, which is consistent with the essential role of KIF20A in cell divisions of NPCs or other stem/progenitor cells (germline knockout of the Kif20a leads to embryonic/perinatal lethality). On the other hand, in silico studies of cancer expression data or expression analyses of clinical samples showed that KIF20A expression is positively correlated with poor prognosis of patients in different types of cancers¹⁶⁻¹⁹. Accordingly, disruption of KIF20A function in brain tumor-initiating cells is demonstrated herein to promote the affected cells to leave the cell cycle and become post-mitotic, and as a result, this should lead to inhibition of brain tumor initiation and/or progression.

As demonstrated in the working examples, a genetic model of MB induced by sustained activation of the Sonic Hedgehog (SHH) pathway was employed. In this model system, inducible knockout of the Patched (Ptc) gene in cerebellar granule neuron progenitors (GNPs) (via expression of CreER under the control of the Atoh1 promoter) results in MB formation within a few months²⁰. Because KIF20A is a mitotic protein that should be expressed by NPCs present in other central nervous system (CNS) areas beyond the cerebral cortex, KIF20A might play a similar role in regulating cell division mode both in normal cerebellar GNPs during development and in tumor-initiating GNPs during tumor formation. To target KIF20A in cerebellum, compound mice carrying Atoh1-CreER, Kif20a^{fl/fl} and Atoh1-CreER, Kif20a^{fl/fl}, Ptc^{fl/fl} alleles were established. Deletion of the Kif20a gene alone or deletion of both the Ptc and Kif20a genes in cerebellar GNPs was investigated. The data obtained from these experiments show that loss-of-function (LOF) of KIF20A resulted in early cell cycle exit and precocious neuronal differentiation in both normal and tumor-initiating GNPs. The data from targeting KIF20A function in human MB cells both in vitro and in vivo is also shown.

In mouse spontaneous brain tumor models, accumulating evidence from genetic studies of MB²⁰⁻²⁵ or glioma²⁶⁻³⁰ have demonstrated that brain tumors originate from dysregulated stem or progenitor cells. Gene expression profiling analyses have also revealed that brain tumor cell transcriptomes display molecular characteristics resembling neural stem/progenitor cell types³¹⁻³⁷. Targeting the proliferation or differentiation programs employed by stem/progenitor cells can effectively interfere with brain tumor initiation or growth. Along these lines, the daughter cells' fate specification process during stem/progenitor cell divisions presents a particularly good opportunity for therapeutic intervention, because the process can occur during the late stages of mitosis and lies downstream of many proliferation-promoting (oncogenic) signaling pathways. As demonstrated herein, the genetic analyses of KIF20A in both normal and cancer-initiating cerebellar GNPs presents an experimental validation of a cell division mode regulator as a potential target for brain tumor inhibition.

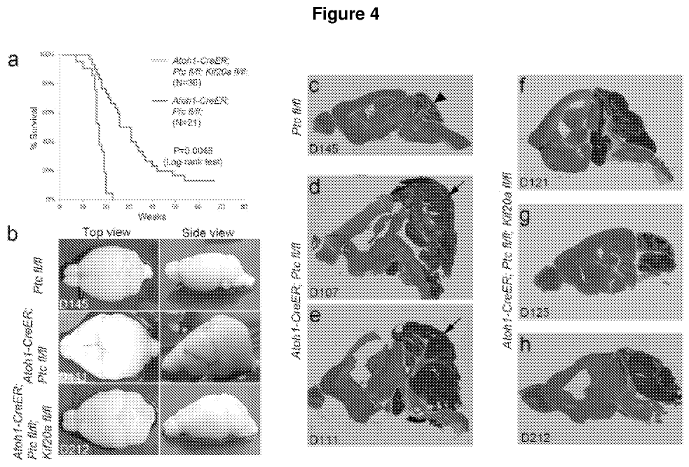

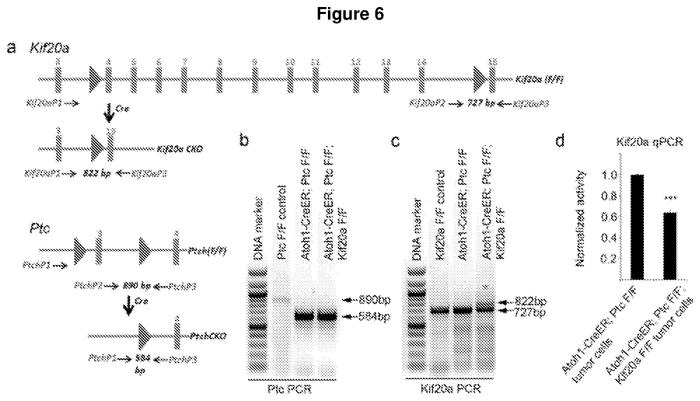

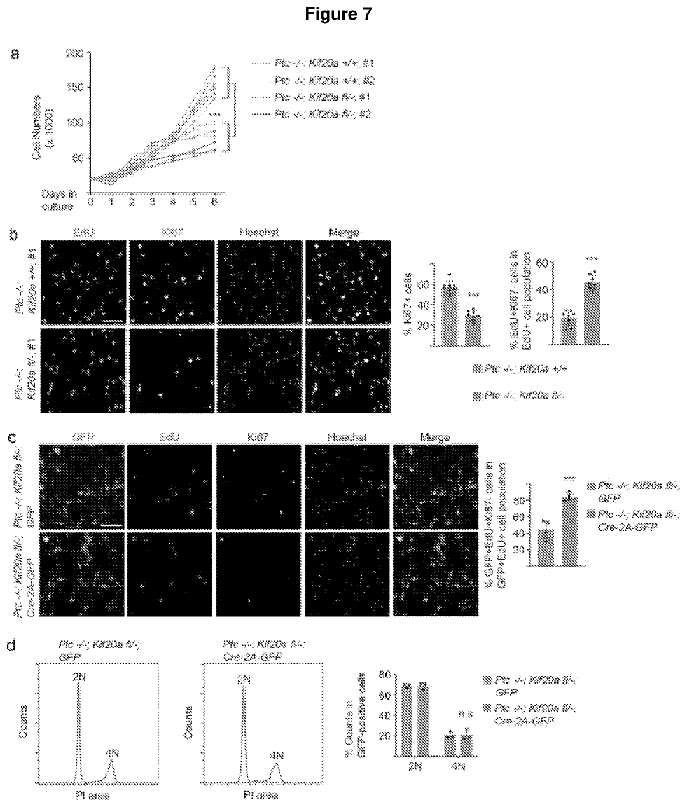

The genetic analyses show that the MB cells isolated from the Atoh1-CreER; Ptc^{fl/fl}; Kif20a^{fl/fl} double knockout mice were homozygous null for the Ptc gene but heterozygous for the Kif20a gene. This result suggests that in the Atoh1-CreER; Ptc^{fl/fl}; Kif20a^{fl/fl} knockout mice, some GNPs responded to tamoxifen induction with one Kif20a allele being deleted but leaving the other Kif20a allele intact. One possible reason for this phenomenon might be that Cre recombinase had lower recombination efficiency at the Kif20a locus than at the Ptc locus. This could be due to some structural hindrance of the Kif20a gene; for instance, 10 exons were floxed in the Kif20a gene but only one exon was floxed in the Ptc gene (FIG. 6a). Another reason might be that Cre-mediated recombination could generate mosaic patterns of recombination products between the Ptc and Kif20a genes, such as cells with genotypes of Ptc^{-/-}; Kif20a^{fl/-} or Ptcd^{fl/-}; Kif20a^{-/-} or other combinations. The GNPs of the Ptc^{-/-}; Kif20a^{fl/-} genotype were detected in tumor cells because they should have had an advantage in proliferation over GNPs of other combinations of genotypes, and as a result, they eventually grew out into a tumor over the time. Regardless of how the Ptc^{-/-}; Kif20a^{fl/-} GNPs were produced, the results indicate that the prolonged survival time in the tamoxifen-induced Atoh1-CreER; Ptc^{fl/fl}; Kif20a^{fl/fl} mice (FIG. 4a) might be underestimated, due to the incomplete deletion of the Kif20a gene in some of the cancer-initialing progenitor cells. Thus, further deletion of the remaining allele of the Kif20a gene in tumor cells derived from the Ptc/Kif20a double knockout mice (cells having a genotype of Ptc^{-/-}; Kif20a^{fl/-}) accelerated cell cycle exit (FIG. 7c).

The data obtained from Atoh1-CreER; Kif20a^{fl/fl} single and Atoh1-CreER; Ptc^{fl/fl}; Kif20a^{fl/fl} double knockout mice together show that KIF20A functions similarly in normal and cancer-initiating GNPs to maintain their proliferative potential. It was previously found that blocking KIF20A function in NPCs of the developing cerebral cortex did not compromise the divisions of parental progenitor cells but drive the daughter cells to adopt a differentiative fate⁹. Data from this study indicates that GNPs with LOF of KIF20A were able to complete cell divisions but the nascent daughter cells became differentiated into neurons, leading to early depletion of proliferating cells in the developing cerebellum as well as in MB. These results thus indicate that targeting KIF20A, or other regulators of cell division mode control in a broad sense, is distinct from conventional anti-mitotic inhibitors, which act primarily to induce mitotic arrest or cytokinesis defect. Blocking the function of a cell division mode regulator does not disrupt a progenitor cell's division process per se but promotes the daughter cells to take a differentiative path. Targeting the process of daughter cell fate specification may thus present a new strategy for expanding the repertoire of anti-proliferation chemotherapy for malignant brain tumors.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Examples

### Materials and Methods

Antibodies: KIF20A(L-13) (Santa Cruz, sc-104954); KIF20A (OriGene, AP01361 PU-N); Alpha-tubulin(TU-01) (ThermoFisher, MA1-19162); Ki67(SP6) (Abcam, Ab16667); Active caspase 3 (BD Biosciences, 559565); NeuN (Millipore, MAB377); Secondary antibodies were purchased from Jackson ImmunoResearch Laboratories (Cy3, Cy5 and Cy2 AffiniPure conjugated); Click-iT®EdU Alexa Fluor®594 image kit (ThermoFisher Scientific).

DNA Constructs, Lentivirus Production: Lentiviral transfer plasmids used in this study include: Hiv7CMV-Cre-myc-2A-GFP (Addgene #117148), Hiv7CMV-GFP, Hiv7CMV-GFP-IRES-Luciferase, as well as Tet-on inducible shRNA plasmids Tet-pLKO-hushKIF20A₇₂₆ or Tet-pLKO-hushKIF20A₁₈₁-Puro (shRNA sequences were cloned into pLKO-Tet-On plasmid, Addgene #21915 at Agel/EcoRI sites). The plasmids used for shRNA Luciferase in vitro screening include: pNUTS-shScramble (Geng et al., 2018), pNUTS-shhuKIF20A₇₂₆ or pNUTS-shhuKIF20A₁₈₁ and shRNA template plasmid pSiCheck2.2-huKIF20A-CDS. Lentiviruses were generated by co-transfecting 293T cells with 15 μg Lentiviral transfer plasmids, 15 μg second generation lentiviral packaging plasmid psPAX2 (Addgene #12260) and 5 μg lentiviral envelope plasmid pCMV-G in 10 cm tissue culture plate using calcium phosphate cell transfection reagents. The growth media were exchanged around 6 hours later and lentivirus-containing supernatant was harvested 48 hours later.

Mice: Kif20a^{fl/fl} mice were deposited in Mutant Mouse Resources and Research Centers (MMRRC) (strain ID: MMRRC_050513-UCD). Atoh1-CreER; Ptc^{fl/fl} mice were obtained from Dr. Robert Wechsler-Reya's group. The two strains were crossed to generate Atoh1-CreER; Kif20a^{fl/fl} and Atoh1-CreER; Ptc^{fl/fl}; Kif20a^{fl/fl} mice. For analyses of normal and MB-initiating GNPs in knockout animals, all mice were treated with a single dose of tamoxifen by gavage at P4, as described in the SHH-MB model²⁰. Animals were group housed and maintained in the temperature range and environmental conditions recommended by AAALAC. Animal procedures were approved by the Institutional Animal Care and Use Committee (IACUC) of Beckman Research Institute of the City of Hope and were carried out in accordance with NIH guideline and the Guide for the Care and Use of Laboratory Animals.

shRNA design and screening: Potential shRNA sequences were selected using web-based design tool (rnaidesigner.thermofisher.com/rnaiexpress/design.do). shRNAs were expressed under the control of a mouse U6 promoter in pNUTs vector which additionally contains a ubiquitin promoter-EGFP expression cassette. Candidate shRNAs in pNUTs and cDNA target in psi-CHECK were co-transfected into HEK293 cells in triplicates; 48 hours later, the firefly and Renilla Luciferase values were determined with Promega's Dual-Luciferase® Reporter Assay System (Promega, E1910). The final inhibition unit (% luciferase activity) was the normalized value (Renilla/Firefly). The sequences of 19mer human KIF20A shRNAs (the loop of shRNA is underlined) are as below:

Scrambled shRNA:
(SEQ ID NO: 120)
5′-CGGCTGAAACAAGAGTTGG-TTCAAGAGA-
CCAACTCTTGTTTCAGCCG-3′
shKIF20A726:
(SEQ ID NO: 121)
5′-GAGGAGTGTCTACATCGAA-TTCAAGAGA-
TTCGATGTAGACACTCCTC-3′
shKIF20A181:
(SEQ ID NO: 122)
5′-AGTATGGAGAAGGTGAAAG-TTCAAGAGA-
CTTTCACCTTCTCCATACT-3′

Western Blot: Daoy cell clones stably integrated with Tet-on shKIF20A726 were cultured with or without Doxycycline induction for 3 days. These Daoy cells were washed with PBS, lysed with 2×SDS loading buffer and boiled for 5 minutes. Denatured proteins were resolved by SDS-PAGE and transferred to a PVDF membrane for Western blot detection by KIF20A (L13) antibody (1:500) and HRP-conjugated Donkey anti-Goat second antibody (1:2500) with chemiluminescent substrate (ThermoFisher Scientific, Cat: 34095).

Histology, immunocytochemistry and Immunohistochemistry: Animal were perfused with 1×PBS followed by 4% paraformaldehyde (PFA). The whole brains were removed, fixed in 4% PFA overnight, cryoprotected in 30% sucrose and embedded in Tissue Tek O.C.T. Compound (Fisher HealthCare). For histological analysis, whole brain sagittal sections (6 μm) were stained with hematoxylin and eosin (Sigma). For immunohistochemistry, whole brain sagittal sections (16 μm) or fixed tumor cells were blocked and permeabilized for 2 hours at room temperature in blocking solution (1×PBS, 0.1% Triton X-100, 10% Donkey serum, 5% AffiniPure Fab fragment Donkey anti-Mouse IgG, 0.2% Sodium Azide), followed by incubation with primary antibody in blocking solution at 4° C. for overnight. The sections or cells were rinsed three times with 1×PBS and incubated with secondary antibody in 1×PBS for 1 hour at room temperature. Sections or cells were counterstained with Hoechst 33342 dye and rinse three times with 1×PBS followed by once in water, then the sections or cells were mounted with Fluoromount-G (SouthernBiotech) for imaging with microscope.

Analyses of inducible knockout mice: The pups of Atoh1-CreER; Ptc^{fl/fl} and Atoh1-CreER; Ptc^{fl/fl}; Kif20a^{fl/fl} mice were treated with tamoxifen at P4 by oral gavage (0.6 mg/30 μl) using 24 G gavaging needles. Tamoxifen (T5648, Sigma) was prepared as a 20 mg/ml stock solution in corn oil (Sigma). EdU was then given by intraperitoneal injection at P5 with the dosage of 0.1 mg per gram of the pups. After 24 or 48 hours post EdU treatment, the brain samples were collected at P6 and P7 for analyses. Cryosections of P6 or P7 brains (16 m) were analyzed for immunostaining on EdU, Ki67 (1:200), NeuN (1:100), or activated caspase 3 (1:200). Stained sections were mounted with Fluoromount-G for imaging. For MB formation analysis, after pups were treated with tamoxifen at P4 by gavage, they were monitored until symptoms of brain tumor growth occurred. Brain tumor samples were removed and cryosections were made for histological documentation.

For FACS-based cell cycle analysis, pieces of P6 cerebellums were gently dissociated in cold HBSS (Mediatech) with 5 mM EDTA. After centrifugation with 300 g at 4° C. for 10 minutes, cells were washed twice with cold HBSS buffer. Subsequently, large cell/aggregates were removed with a cell strainer (40 μm). After fixation with 70% ethanol drop by drop and then being stored for overnight at 4° C., cells were washed in cold PBS twice. For propidium iodide staining, cells were resuspended in 0.5 ml staining buffer (PBS+10 μg/ml PI+0.1% Triton X-100+0.2 mg/ml DNAse-free RNAse A) and were incubated at 37° C. for 30 minutes. Cells were filtered before FACS analysis. Measurements were done using a CyAn ADP instrument (DakoCytomation) in combination with summit software.

Expansion of mouse MB cells in culture: Brains with MB were dissected out and transferred into PBS on ice. Blood and fat tissues were removed from the tumor mass under a dissecting microscope and the tumor tissue pieces were washed again with PBS. Tumor tissues were minced with a scalpel, passed through syringes with 18- and 22-gauge needles, and then incubated in a 1:1 mixture of Accutase (Sigma) and TrypLE (Invitrogen) for 15 minutes at 37° C. with occasionally vortex. Undigested tissues were removed by filtering with 40 μm cell strainer (Falcon, 352340). Dissociated cells were washed twice with DMEM/F12 medium followed by centrifugation at 2000 rpm for 5 minutes. Cells were then re-suspended and plated onto uncoated dishes in Neurobasal and DMEM/F12 media (1:1 mix) containing N2 and B27 supplements (Invitrogen), human recombinant FGF2 and EGF (20 ng/ml, PEPROTECH). Five to seven days later, cell spheres were dissociated in Accutase (Sigma-Aldrich) and plated onto Primaria dishes (BD Biosciences) coated with Poly-L-ornithine solution (Sigma-Aldrich) and mouse laminin (Sigma-Aldrich) to allow adherent growth.

Generation of Daoy cell lines with stably integrated Tet-on inducible shRNA expression system: Daoy cells were infected with lentivirus of Tet-on inducible human KIF20A shRNA-726 and GFP-Luciferase prepared from Tet-pLKO-hushKIF20A₇₂₆ plasmid and Hiv7CMVGFP-IRES-Luciferase plasmid, respectively. The transduced Daoy cells were selected for stable single clones with Puromycin (1 μg/ml, Sigma-Aldrich) and GFP fluorescence signal in DMEM (Invitrogen) with 10% fetal bovine serum. Single clones were picked up by glass cloning cylinders (Sigma-Aldrich) in 10 cm plate. Three clones were obtained and tested for growth ability and doxycycline (0.5 μg/ml) inducibility using growth curve assay, western blots and luciferase reporter assay system. Clone #2 was selected for further use.

Growth Assay of tumor cells: Mouse MB cell lines, or Daoy clones stably integrated with Tet-on inducible human KIF20A shRNA-726, or Wild Type Daoy cells, were seeded into multiple wells of 24 well plates (2×10⁴ or 1×10⁴ cells per well). Cells in quadruplicate were then dissociated and counted using a hemocytometer at consecutive days after initial plating.

Analyses of KIF20A expression using transcriptome databases of MB patient samples: Raw microarray CEL files were obtained from the two published MB cohorts, GSE85217³⁸ and EGAS00001001953³⁹. Expression data was quantified using custom chip definition files corresponding to Ensembl genes (v23.0.0) from brain array⁴⁰ and then each dataset was normalized using robust multichip averaging (RMA) from the oligo package v1.54.1⁴¹. Duplicate samples were removed and then datasets were integrated for batch-correction using ComBat from the sva package v3.36.0⁴².

Intracranial tumor transplantation and Bioluminescence imaging: NSG mice (8-10 weeks old) were anesthetized with isoflurane and oxygen and placed under microscope. After exposing the skull with a scalpel, a cell suspension (1×10⁵ cells in 4 μl PBS) of Daoy clone #2 stably integrated with Tet-on inducible human KIF20A shRNA (shRNA726) and Luciferase gene was slowly injected into the cerebellum at a depth of 2.5 mm using a 10 μl Hamilton syringe with a 26 G needle using a plastic blocker to control the injection depth. After injection, the incision was closed using wound clips. The transplanted mice were then monitored weekly with luciferase-based bioluminescence imaging for tumor growth. When tumor growth was evident (four weeks after cell implantation), the mice were separated into two groups, control group and doxycycline-treatment group. For the treatment group, mice were first gavaged with 0.2 ml of Doxycycline (10 mg/ml) for two consecutive days. Then the mice were fed continuously with Doxycycline-containing food (TestDiet, 625 ppm), with the food being replaced every other day. Tumor growths in two mice groups were monitored weekly by bioluminescence imaging. Survivals of mice after cell transplantation were recorded and analyzed.

Image acquisition and processing: Fluorescent images were taken with Zeiss Observer II or confocal microscope of Zeiss LSM 700 or Zeiss LSM 880.

Statistics and Reproducibility: The numbers of tumor-forming knockout or NSG mice (animal survival experiment), brain samples used in each genetic analysis, or replicates of cell samples (wells) in cultured cell experiments, were indicated in the histograms of the figures. All data were presented as mean±standard deviation (SD). Student's t-test or two-way ANOVA analyses were performed and indicated in histograms of the figures. Log-rank test was performed for the Kaplan-Meier curve. In all descriptions, *represented p<0.05; **represented p<0.01; ***represented p<0.001; n.s. represented not significant (p>0.05). Cultured cell-related experiments were repeated twice, and similar results were obtained.

### Example 1 Conditional Knockout of Kif20a in Cerebellar GNPs Caused Early Cell Cycle Exit and Precocious Neuronal Differentiation

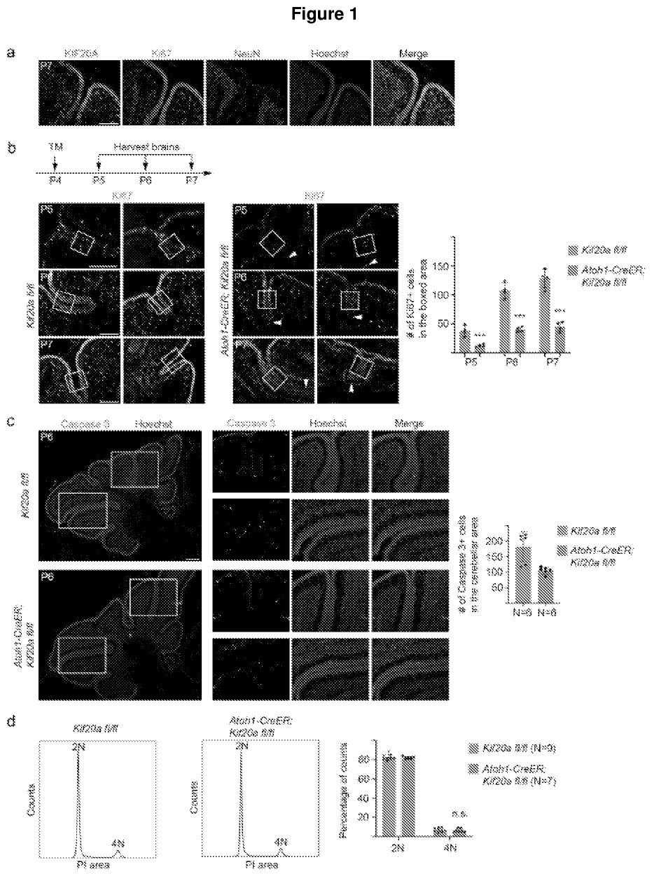

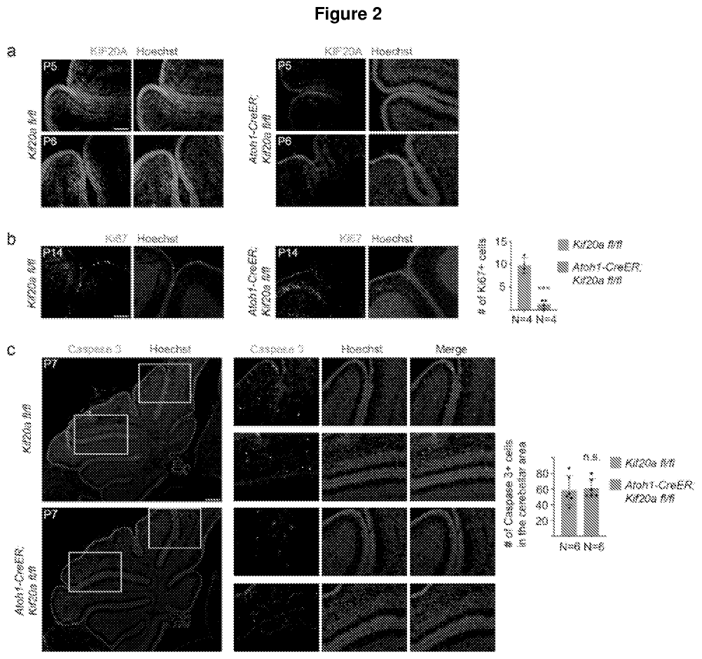

In the mouse cerebellum, immunostaining shows that strong KIF20A expression was enriched within the external granule layer (EGL), where GNPs are located during early postnatal stages (FIG. 1a). To study the potential function of KIF20A in cerebellar development, inducible deletion of the Kif20a gene in GNPs was performed. Conditional Kif20a knockout mice (littermates of Atoh1-CreER; Kif20a^{fl/fl} and control Kif20a^{fl/fl} mice) were treated with tamoxifen at postnatal day 4 (P4). Brain samples were then collected from P5 for analyses. Assessment of KIF20A expression in the P5 and P6 samples indicated that deletion of Kif20a was evident one day after tamoxifen induction (FIG. 2a). Examination of Ki67 expression in the brain samples revealed a loss of Ki67⁺ cells in the mutant cerebellums following the induced deletion of Kif20a (FIGS. 1b and 2b). To address whether the loss of proliferating cells resulted from increased death of mutant GNPs (due to possible defect in cytokinesis after Kif20a knockout), immunostaining for activated caspase 3 was performed. The results from P6 and P7 brain samples show that the apoptosis levels in the cerebellums were overall low and comparable between the Kif20a knockout and wild-type littermate brains (FIGS. 1c and 2c). It was previously found that knockout of Kif20a did not cause a noticeable failure of cytokinesis in neural progenitor divisions of the developing cerebral cortex⁹. To assess the status of cytokinesis process in the mutant cerebellums, flow cytometry-based cell cycle analysis was performed. The results show that the 4N DNA contents between the P6 mutant (Atoh1-CreER; Kif20a^{fl/fl}) and control (Kif20a^{fl/fl}) cerebellar cells were comparable (FIG. 1d), indicating that knockout of Kif20a did not cause an obvious defect in cytokinesis of GNP divisions, or a possible cytokinesis defect was largely compensated by other factors.

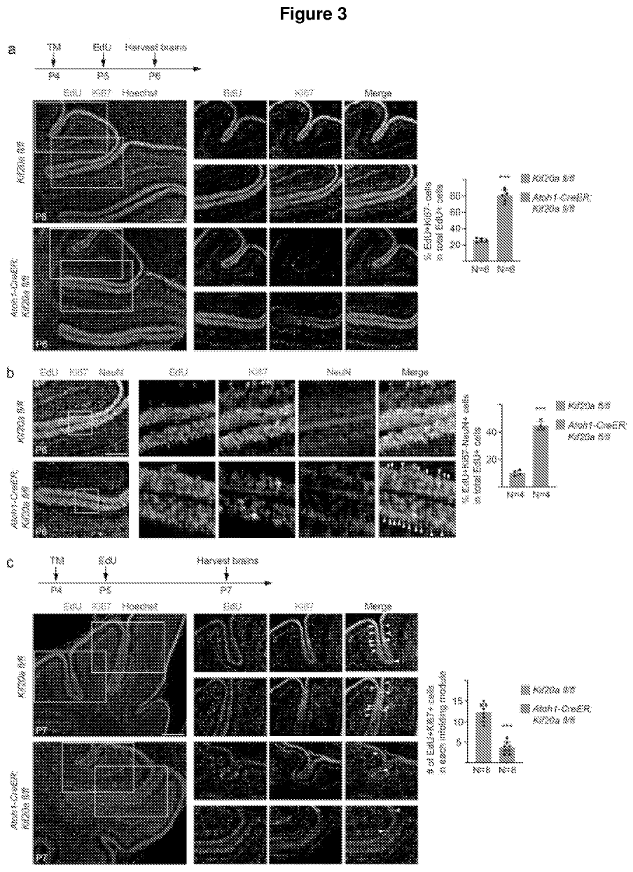

Next the cell cycle exit and re-entry in GNPs of the Kif20a knockout cerebellum were examined. In this experiment, conditional Kif20a mice were treated with tamoxifen at P4 followed by an injection of EdU at P5, and brain samples were then collected at P6 or P7 for analyses (FIG. 3). In the P6 brains, co-staining of EdU and Ki67 showed that within individual cerebellar sulci, there were relatively more EdU⁺Ki67⁻ cells (cells having left the cell cycle after being labeled by EdU) in the mutant EGL than in the wild-type littermate EGL (FIG. 3a). Co-staining with neuronal marker NeuN further shows that more EdU⁺Ki67⁻ cells in the Kif20a knockout brains were positive for NeuN (FIG. 3b). These EdU⁺Ki67⁻ NeuN+ cells were located outside the proliferating zone (Ki67⁺ cell zone) (FIG. 3b), reflecting developmental progression of differentiated granule neurons emanating from the EGL. This result also suggests that knockout of Kif20a did not compromise the completion of cell division (cytokinesis) of the mutant GNPs. In the P7 brains, within individual cerebellar sulci, significantly fewer EdU⁺Ki67⁺ cells (cells remaining in the cell cycle) could be seen in the mutant EGL than in the wild-type EGL (FIG. 3c). These data collectively suggest that KIF20A is essential for maintaining the proliferative state of GNPs and that LOF of KIF20A in GNPs causes cell cycle exit and leads to an early depletion of the progenitor population.

### Example 2 Blocking KIF20A Function Inhibited SHH-Induced MB

To address whether KIF20A might be similarly crucial for maintaining the proliferation of brain tumor-initiating cells, the Kif20a^{fl/fl} floxed mice were crossed with Atoh1-CreER; Ptc^{fl/fl} mice to generate a strain of compound mice carrying the Atoh1-CreER; Ptc^{fl/fl}; Kif20a^{fl/fl} alleles. When tamoxifen was given to the pups at P4, Ptc knockout mice with intact Kif20a (Atoh1-CreER; Ptc^{fl/fl}) developed MB with 100% penetrance within about five months (FIG. 4a), as previously reported²⁰. Ptc/Kif20a double-knockout mice (Atoh1-CreER; Ptc^{fl/fl}; Kif20a^{f}/l), on the other hand, showed a longer survival time before symptoms occurred and some of the mice appeared normal even one-year after tamoxifen induction (FIG. 4a). Among the mice that developed symptoms, the brains of the Ptc/Kif20a double-knockout mice had smaller tumors than those seen in Ptc knockout mice with intact Kif20a (FIGS. 4b-4h). The brain samples from the Ptc/Kif20a double knockout mice generally also retained more normal morphology than those Ptc single-knockout mice (FIGS. 4b and 4f-4h vs. FIGS. 4d-4e). These results suggest that KIF20A is crucial for the development of SHH-induced MB.

### Example 3 LOF of KIF20A in MB-Initiating GNPs Caused Early Cell Cycle Exit

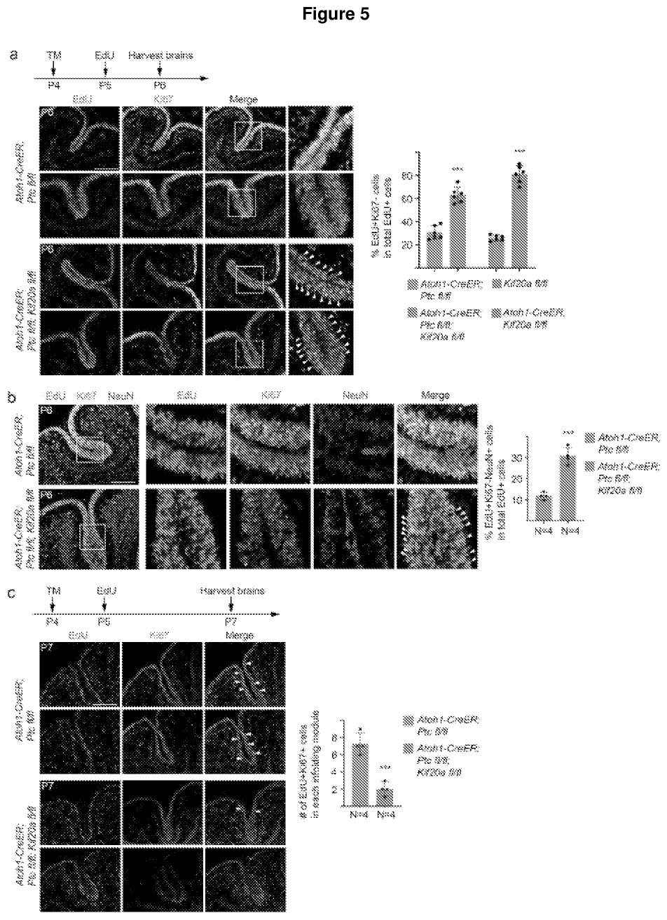

To study the possible mechanism of inhibition of SHH-induced MB by LOF of KIF20A, the cell cycle exit and reentry in tumor-initiating GNPs of the Ptc single and Ptc/Kif20a double knockout mice were examined. The pups of Atoh1-CreER; Ptc^{fl/fl} and Atoh1-CreER; Ptc^{fl/fl}; Kif20a^{fl/fl} mice were treated with tamoxifen at P4 followed by EdU labeling at P5. Brain samples were then collected at P6 and P7 for analysis. In the P6 brains, data from Ki67 staining showed that there were overall fewer Ki67⁺ cells in the cerebellar sulci of the Ptc/Kif20a double knockout brains than those of the Ptc single knockout brains (FIG. 5a). Co-staining of EdU and Ki67 shows that there were more EdU⁺Ki67⁻ cells in the Ptc/Kif20a double knockout EGL than in the Ptc single knockout EGL (FIG. 5a), suggesting that more GNPs in the Ptc/Kif20a double knockout brains had left the cell cycle earlier. Co-staining with neuronal marker NeuN further shows that more EdU⁺Ki67⁻ cells in the Ptc/Kif20a double knockout brains were positive for NeuN and these EdU⁺Ki67⁻ NeuN⁺ cells formed a line surrounding the proliferating zone, reflecting newly born granule neurons exiting the EGL (FIG. 5b). In the P7 brains, fewer proliferating cells (EdU⁺Ki67⁺ cells) remained in the EGL of the Ptc/Kif20a double knockout mice than in the Ptc single knockout EGL (FIG. 5c). These data collectively indicate that KIF20A is also essential for maintaining the proliferative state of tumor-initiating GNPs.

### Example 4 LOF of KIF20A in Mouse Tumor Cells LED to Inhibition of Proliferation by Inducing Differentiation

To further examine the mechanisms of KIF20A function in MB, proliferating cells from MBs derived from both the Atoh1-CreER; Ptc^{fl/fl} and Atoh1-CreER; Ptc^{fl/fl}; Kif20a^{fl/fl} knockout mice were isolated. Some tumor cells could expand in culture and two cell lines from each tumor type were established. CreER-mediated gene deletion was examined by genotyping PCR on genomic DNAs first (FIG. 6a). The results show that targeted homozygous deletion of the Ptc gene was evident in all four tumor cell lines, consistent with the occurrence of MB in these mice (FIG. 6b; results of one cell line from each tumor type were shown). Surprisingly, however, in the cell lines derived from the Ptc/Kif20a double knockout mice, the Kif20a gene was in a heterozygous state, with one allele having the intact floxed Kif20a gene and the other allele having the floxed exons removed (FIG. 6c). Further qPCR on RNAs isolated from these cells shows that a Kif20a transcript was expressed, albeit in a reduced level (FIG. 6d). In theory, tamoxifen-induced activation of CreER should have led to simultaneous homozygous deletion of both the Ptc and Kif20a genes in GNPs carrying the Atoh1-CreER; Ptc^{fl/fl}; Kif20a^{fl/fl} alleles; however, the results suggest that simultaneous deletion of the Kif20a and Ptc genes was faulty in some GNPs of the double knockout mice, such that while the Ptc gene was deleted, the Kif20a gene was left half intact. Thus, the cell lines derived from tumors of the Ptc single and Ptc/Kif20a double knockout mice had the genotype of Ptc^{-/-}; Kif20a^{+/+} and Ptc^{-/-}; Kif20a^{fl/-}, respectively.

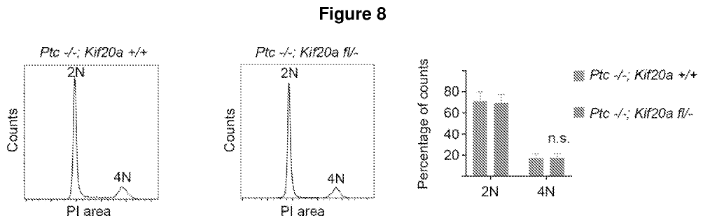

Next, analyses on cell biological properties of these two types of tumor cells were performed. The results show that tumor cells derived from the Ptc/Kif20a double knockout mice had a slower proliferation rate (FIG. 7a), consistent with the overall longer survival time of the double knockout mice (FIG. 4a). These tumor cells also showed fewer Ki67⁺ proliferating cells and were prone to exiting the cell cycle (FIG. 7b), compared to tumor cells derived from the Ptc single knockout mice. The two types of tumor cells displayed comparable 4N DNA contents in cell cycle analysis (FIG. 8), suggesting that deletion of Kif20a in SHH-MB cells did not alter the cytokinesis status. To further test the effect of Kif20a deletion, introduced Cre enzyme (Cre-2A-GFP), or control GFP, was introduced into the tumor cells derived from the Ptc/Kif20a double knockout mice, using a lentiviral expression system. The infected cells were next labeled with EdU for 24 hours before being fixed for immunostaining. The results show that expression of Cre in these heterozygous Kif20a tumor cells caused an increase of EdU⁺Ki67⁻ cells compared to cells expressing GFP control alone (FIG. 7c), suggesting more cells exited the cell cycle after Cre expression. In addition, cell cycle analysis of these tumor cells showed that the cells expressing Cre or control GFP displayed comparable 4N DNA contents (FIG. 7d). These results thus collectively indicate that knocking out the remaining Kif20a allele in the tumor cells derived from the Ptc/Kif20a double knockout mice did not cause an obvious failure in cell division but led to increased exit of the cell cycle in daughter cells. Therefore, the slower growth rate of these tumor cells (FIG. 7a) might be attributed to their tendency for becoming post-mitotic cells due to the half dosage of Kif20a expression, which eventually leads to fewer proliferating cells in the population.

### Example 5 Inducible Knockdown of KIF20A in Human MB Cells Inhibited Proliferation in Culture and Tumor Growth in Xenograft

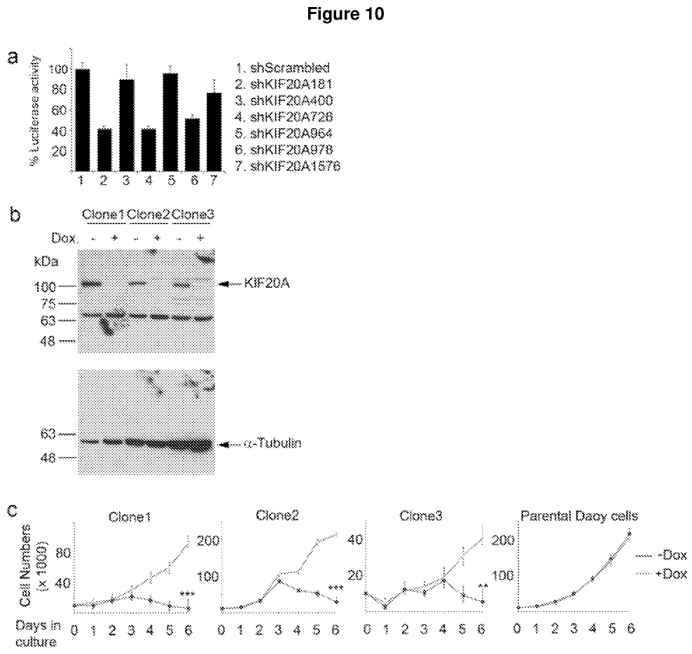

The expression status of KIF20A in relation to human MB cells was examined using available expression data of patient tumor samples. The results revealed that strong KIF20A expression was positively correlated with Ki67 level in different subgroups or subtypes of MBs (FIGS. 9a-9c), indicating that KIF20A is positively associated with proliferation of human MB cells. To address the function of KIF20A in human MB cell proliferation, multiple shRNAs targeting human KIF20A transcript were generated (FIG. 10a). One such shRNA (shKIF20A726) was cloned into a Tet-on inducible shRNA expression vector²¹. The inducible shKIF20A-726 expression cassette and a GFP/firefly luciferase (ffluc) expression cassette were then stably integrated into Daoy human MB cells. Three puromycin-resistant cell clones (stably integrated with Tet-on shKIF20A-726 and GFP/ffluc) were isolated. The data show that doxycycline treatment was able to induce significant knockdown of KIF20A expression in all three cell clones (FIG. 10b). Doxycycline induction was also seen to significantly inhibit the proliferation of all three clones (FIG. 10c). This inhibition was not due to cytotoxicity of doxycycline, because parental Daoy cells were not affected by doxycycline treatment (FIG. 10c). Among the three clones, Clone #2 was chosen for further in vivo studies, as it showed comparable growth rate to the parental cells, indicating minimal alteration of cell properties from lentiviral integration and/or minimal leakage of the Tet-on shKIF20A cassette.

To examine the function of KIF20A in tumor formation by Daoy cells, 10⁵ Clone #2 cells were injected into the cerebellum of each recipient NSG mouse using a method previously described²². Growth of tumor cells was then monitored by bioluminescence imaging. Four weeks after the initial cell injection, tumor growths in the brains of recipient mice were evident by the increase of imaging signal (FIG. 9d). The injected mice were then separated into two groups, one group was treated with doxycycline and the other group was un-treated as control. For the treatment group, the mice were first fed with doxycycline solution (10 mg/ml, 0.2 ml) by gavage daily for two consecutive days. The mice were then kept on doxycycline-containing food continuously and were monitored for tumor growth by bioluminescence imaging. The results revealed that, comparing to the control group, the group of mice received treatment showed significantly slower growth of brain tumors (FIG. 9d) and better overall survival (FIG. 9e), indicating that inhibition of KIF20A expression could suppress cell proliferation in a growing tumor.

### Example 6 KIF20A Knockdown in Patient-Derived Tumor Cells Inhibited Proliferation by Inducing Cell Cycle Exit

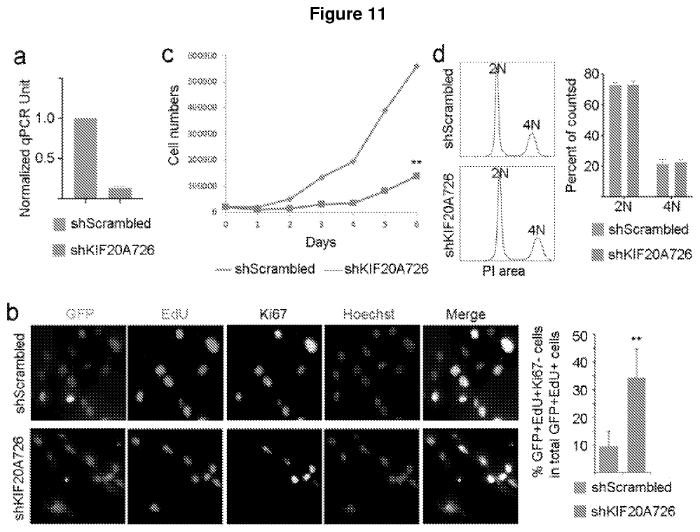

To further investigate the relevance of KIF20A in human brain tumors, inhibition of KIF20A in patient-derived GBM-initiating/stem cells (GISCs) was examined. As the ERKS axis functions in different types of brain tumor-initiating/stem cells, data obtained from study of GISCs would be applicable to other brain tumor cell types. Multiple lines of patient-derived GISCs were obtained from Dr. Christine Brown. These low passaged GISCs could be expanded in cultures and could engraft in NSG mice^{43, 44}. FIG. 11 summarizes the results of testing KIF20A in one GISC line (GISC017) by constitutive expression of a control scrambled shRNA or shKIF20A726 (shRNA targeting hKIF20A) in pNUT vector-based lentiviral expression system. Compared to the scrambled shRNA control, expression of shKIF20A726 in GISC017 cells strongly knocked down KIF20A level (FIG. 11a), resulted in more cells exiting the cell cycle (FIG. 11b) and a slower cell growth rate (FIG. 11c). In the above cell cycle exit analysis, co-staining of differentiation markers (e.g. βlll-tubulin, DCX, or NeuN) was performed, however, these markers were detected in EdU⁺Ki67⁻ (cells having left the cell cycle) but also in some EdU⁺Ki67⁺ (cells staying in the cell cycle) cells, which might reflect a technical artefact observed with progenitor/stem cells in culture. While this precluded an analysis of the differentiation state of the GISCs that have KIF20A depleted, based on the observation that LOF of KIF20A in endogenous MB-initiating GNPs caused precocious neuronal differentiation (FIG. 5b), the observed cell cycle exit of GISCs (FIG. 11b) was an indication that LOF of KIF20A could induce differentiation of GISCs. Further flow cytometry-based cell cycle analysis showed that the 4N DNA contents between cells expressing shKIF20A726 and cells expressing scrambled shRNA control were comparable (FIG. 11d), suggesting there was no obvious cytokinesis defect due to KIF20A knockdown.

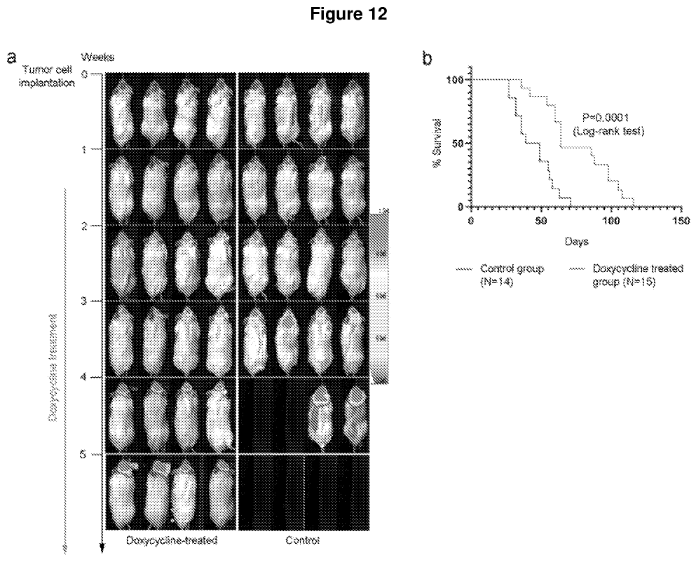

GISC017 cell clones stably integrated with a Tet-inducible shKIF20A726 and luciferase expression cassette were established. When these cells were transplanted into the brains of recipient NSG mice, doxycycline treatment could suppress tumor xenograft growth and prolong animal survival (FIG. 12).

### Example 7 Inhibition of RGS3-KIF20A Interaction in Patient-Derived Tumor Cells Promoted Cell Cycle Exit

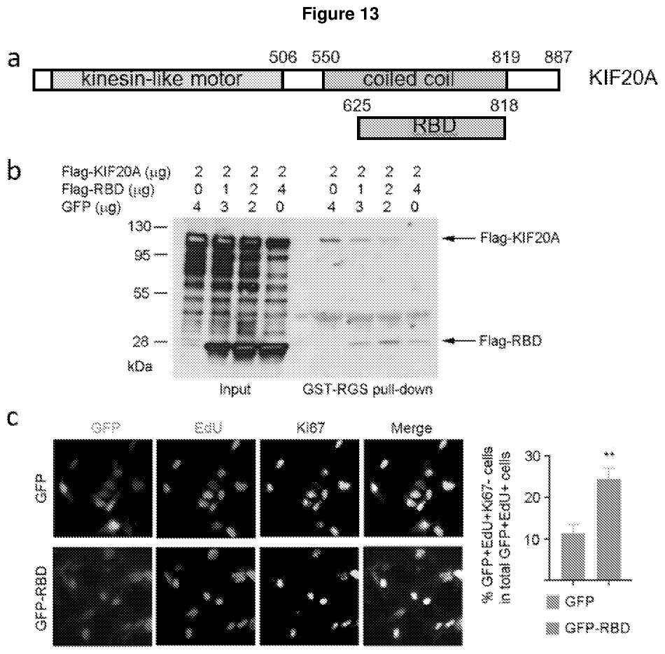

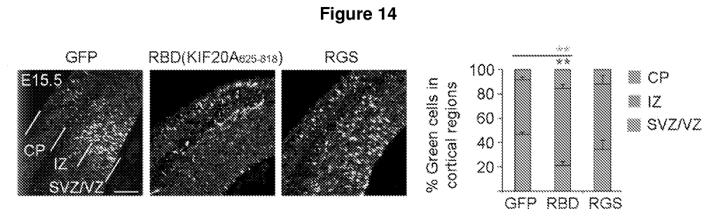

RGS3-KIF20A interaction is mediated by the RGS domain of RGS3 and the RGS-binding domain (RBD) of KIF20A which is located to amino acids 625-818 of mouse KIF20A sequence (FIG. 13a)⁹. Further deletions to the RBD domain resulted in diminished binding capacity compared to the intact RBD(KIF20A₆₂₅-818) (unpublished data). In a GST-RGS pulldown experiment, it was found that expression of Flag-RBD inhibited binding of GST-RGS and KIF20A in a dose dependent manner (FIG. 13b), demonstrating that the RBD is a competitive inhibitor for RGS3-KIF20A interaction. Interestingly, in utero electroporation (IUE)-based over-expression of the RBD in the mouse cortex caused precocious neuronal differentiation of the affected NPCs (FIG. 14). In this experiment, expression plasmid of GFP, RBD (RGS-binding domain of KIF20A), or RGS was delivered into the cortices at E13.5 via IUE. Brains were collected at E15.5 for analyses. Distribution of transfected cells (GFP+) across the radial domains were scored. FIG. 14 shows that expression of mouse RBD (KIF20A₆₂₅₋₈₁₈) caused neural progenitor cell differentiation and subsequent outward migration of newly born neurons into the IZ and CP regions. In addition, when expressed in GISC017 cells by lentiviral infection, the RBD could induce cell cycle exit (FIG. 13c). These preliminary data suggested that the RGS3-KIF20A interaction is crucial for proliferation vs. differentiation control in GISCs and that it may serve as a target for inhibition of brain tumor cell proliferation.

### Example 8 Inhibition of EphrinB-RGS3 Interaction in Patient-Derived Tumor Cells Promoted Cell Cycle Exit and Inhibited Cell Proliferation

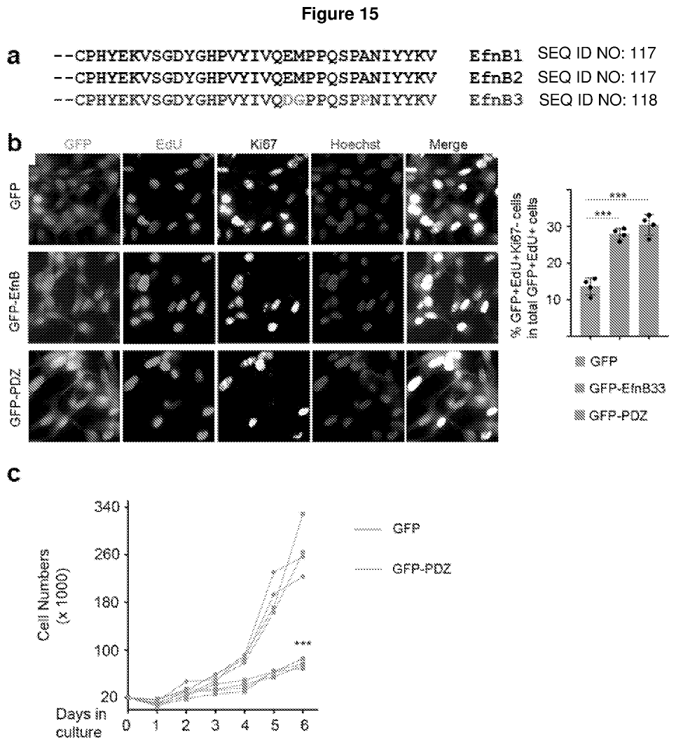

Interaction between EphrinB and RGS3 is mediated by the PDZ domain of RGS3 and the C-terminal sequence of EphrinB, which is highly conserved among all three known EphrinB family members (FIG. 15a). The PDZ domain and the C-terminus of EphrinB may function as competitive inhibitors to disrupt the function of EphrinB-RGS3 interaction in stem/progenitor cells. It was previously observed that over-expression of the PDZ domain (as a GFP fusion protein PDZ-GFP) in the embryonic mouse cortex could cause precocious neuronal differentiation in NPCs⁴⁵, suggesting that the PDZ domain could interfere with EphrinB-RGS3 interaction and function. To address whether the PDZ domain or C-terminus of EphrinB could act similarly in tumor cells, the effect of over-expression of these peptides in brain tumor cells was investigated. Preliminary data showed that lentivirus-based over-expression of the PDZ domain or the conserved C-terminal 33 amino acid sequence of EphrinB promoted cell cycle exit and suppress proliferation in Daoy human medulloblastoma cells (data not shown) and patient-derived glioblastoma-initiating/stem cells (GISCs) (FIGS. 15b, 15c), indicating that the PDZ domain of RGS3 or the EphrinB C-terminus peptide could be used as competitive inhibitors to suppress brain tumor cell proliferation.

### REFERENCES

The references, patents and published patent applications listed below, and all references cited in the specification above are hereby incorporated by reference in their entireties, as if fully set forth herein.
1. Caussinus E, Gonzalez C. Induction of tumor growth by altered stem-cell asymmetric division in Drosophila melanogaster. Nature genetics 37, 1125-1129 (2005).
2. Betschinger J, Mechtler K, Knoblich J A. Asymmetric segregation of the tumor suppressor brat regulates self-renewal in Drosophila neural stem cells. Cell 124, 1241-1253 (2006).
3. Lee C Y, Wilkinson B D, Siegrist S E, Wharton R P, Doe C Q. Brat is a Miranda cargo protein that promotes neuronal differentiation and inhibits neuroblast self-renewal. Developmental cell 10, 441-449 (2006).
4. Lee C Y, et al. Drosophila Aurora-A kinase inhibits neuroblast self-renewal by regulating aPKC/Numb cortical polarity and spindle orientation. Genes & development 20, 3464-3474 (2006).
5. Wang H, Somers G W, Bashirullah A, Heberlein U, Yu F W, Chia W. Aurora-A acts as a tumor suppressor and regulates self-renewal of Drosophila neuroblasts. Genes & development 20, 3453-3463 (2006).
6. Wang H Y, Ouyang Y S, Somers W G, Chia W, Lu B W. Polo inhibits progenitor self-renewal and regulates Numb asymmetry by phosphorylating Pon. Nature 449, 96-U70 (2007).
7. Gonzalez C. Spindle orientation, asymmetric division and tumour suppression in Drosophila stem cells. Nature reviews Genetics 8, 462-472 (2007).
8. Neumuller R A, Knoblich J A. Dividing cellular asymmetry: asymmetric cell division and its implications for stem cells and cancer. Genes & development 23, 2675-2699 (2009).
9. Geng A, et al. KIF20A/MKLP2 regulates the division modes of neural progenitor cells during cortical development. Nature communications 9, 2707 (2018).
10. Qiu R X, et al. SEPT7 Interacts with KIF20A and Regulates the Proliferative State of Neural Progenitor Cells During Cortical Development. Cerebral Cortex 30, 3030-3043 (2020).
11. Dirks P B. Brain tumor stem cells: The cancer stem cell hypothesis writ large. Mol Oncol 4, 420-430 (2010).
12. Chen J, Mckay R M, Parada L F. Malignant Glioma: Lessons from Genomics, Mouse Models, and Stem Cells. Cell 149, 36-47 (2012).
13. Wang J, Wechsler-Reya R J. The role of stem cells and progenitors in the genesis of medulloblastoma. Experimental neurology 260, 69-73 (2014).
14. Fine H A. New Strategies in Glioblastoma: Exploiting the New Biology. Clin Cancer Res 21, 1984-1988 (2015).
15. Gimple R C, Bhargava S, Dixit D, Rich J N. Glioblastoma stem cells: lessons from the tumor hierarchy in a lethal cancer. Genes & development 33, 591-609 (2019).
16. Kawai Y, et al. KIF20A expression as a prognostic indicator and its possible involvement in the proliferation of ovarian clearcell carcinoma cells. Oncology reports 40, 195-205 (2018).
17. Sheng Y, et al. Upregulation of KIF20A correlates with poor prognosis in gastric cancer. Cancer management and research 10, 6205-6216 (2018).
18. Zhao X, et al. Overexpression of KIF20A confers malignant phenotype of lung adenocarcinoma by promoting cell proliferation and inhibiting apoptosis. Cancer medicine 7, 4678-4689 (2018).
19. Zhou Y M, et al. Identification of Potential Biomarkers in Glioblastoma through Bioinformatic Analysis and Evaluating Their Prognostic Value. BioMed research international 2019, (2019).
20. Yang Z J, et al. Medulloblastoma can be initiated by deletion of Patched in lineage-restricted progenitors or stem cells. Cancer cell 14, 135-145 (2008).
21. Wiederschain D, et al. Single-vector inducible lentiviral RNAi system for oncology target validation. Cell cycle (Georgetown, Tex 8, 498-504 (2009).
22. Ozawa T, James C D. Establishing intracranial brain tumor xenografts with subsequent analysis of tumor growth and response to therapy using bioluminescence imaging. Journal of visualized experiments: JoVE, (2010).
23. Schuller U, et al. Acquisition of granule neuron precursor identity is a critical determinant of progenitor cell competence to form Shh-induced medulloblastoma. Cancer cell 14, 123-134 (2008).
24. Grammel D, et al. Sonic hedgehog-associated medulloblastoma arising from the cochlear nuclei of the brainstem. Acta neuropathologica 123, 601-614 (2012).
25. Li P, et al. A population of Nestin-expressing progenitors in the cerebellum exhibits increased tumorigenicity. Nature neuroscience 16, 1737-1744 (2013).
26. Holland E C, Celestino J, Dai C K, Schaefer L, Sawaya R E, Fuller G N. Combined activation of Ras and Akt in neural progenitors induces glioblastoma formation in mice. Nature genetics 25, 55-57 (2000).
27. Bachoo R M, et al. Epidermal growth factor receptor and Ink4a/Arf: Convergent mechanisms governing terminal differentiation and transformation along the neural stem cell to astrocyte axis. Cancer cell 1, 269-277 (2002).
28. Uhrbom L, Kastemar M, Johansson F K, Westermark B, Holland E C. Cell type-specific tumor suppression by Ink4a and Arf in kras-induced mouse gliomagenesis. Cancer research 65, 2065-2069 (2005).
29. Zhu Y, et al. Early inactivation of p53 tumor suppressor gene cooperating with NF1 loss induces malignant astrocytoma. Cancer cell 8, 119-130 (2005).
30. Alcantara Llaguno S, et al. Malignant astrocytomas originate from neural stem/progenitor cells in a somatic tumor suppressor mouse model. Cancer cell 15, 45-56 (2009).
31. Patel A P, et al. Single-cell RNA-seq highlights intratumoral heterogeneity in primary glioblastoma. Science (New York, NY 344, 1396-1401 (2014).
32. Lee J H, et al. Human glioblastoma arises from subventricular zone cells with low-level driver mutations. Nature 560, 243-+(2018).
33. Neftel C, et al. An Integrative Model of Cellular States, Plasticity, and Genetics for Glioblastoma. Cell 178, 835-+(2019).
34. Vladoiu M C, et al. Childhood cerebellar tumours mirror conserved fetal transcriptional programs. Nature 572, 67-+(2019).
35. Hovestadt V, et al. Resolving medulloblastoma cellular architecture by single-cell genomics. Nature 572, 74-79 (2019).
36. Jessa S, et al. Stalled developmental programs at the root of pediatric brain tumors. Nature genetics 51, 1702-+(2019).
37. Zhang L G, et al. Single-Cell Transcriptomics in Medulloblastoma Reveals Tumor-Initiating Progenitors and Oncogenic Cascades during Tumorigenesis and Relapse. Cancer cell 36, 302-+(2019).
38. Cavalli F M G, et al. Intertumoral Heterogeneity within Medulloblastoma Subgroups. Cancer cell 31, 737-754 e736 (2017).
39. Northcott P A, et al. The whole-genome landscape of medulloblastoma subtypes. Nature 547, 311-317 (2017).
40. Dai M, et al. Evolving gene/transcript definitions significantly alter the interpretation of GeneChip data. Nucleic Acids Res 33, e175 (2005).
41. Carvalho B S, Irizarry R A. A framework for oligonucleotide microarray preprocessing. Bioinformatics 26, 2363-2367 (2010).
42. Leek J T, Johnson W E, Parker H S, Jaffe A E, Storey J D. The sva package for removing batch effects and other unwanted variation in high-throughput experiments. Bioinformatics 28, 882-883 (2012).
43. Brown C E, Starr R, Martinez C, Aguilar B, D'Apuzzo M, Todorov I, Shih C C, Badie B, Hudecek M, Riddell S R, Jensen M C. Recognition and Killing of Brain Tumor Stem-Like Initiating Cells by CD8(+) Cytolytic T Cells. Cancer research. 2009; 69(23):8886-93. doi: 10.1158/0008-5472.CAN-09-2687. PubMed PMID: WOS:000272362800008.
44. Brown C E, Starr R, Aguilar B, Shami A F, Martinez C, D'Apuzzo M, Barish M E, Forman S J, Jensen M C. Stem-like Tumor-Initiating Cells Isolated from IL13R alpha 2 Expressing Gliomas Are Targeted and Killed by IL13-Zetakine-Redirected T Cells. Clin Cancer Res. 2012; 18(8):2199-209. doi: 10.1158/1078-0432.CCR-11-1669. PubMed PMID: WOS:000302907300011.
45. Qiu R, Wang X, Davy A, Wu C, Murai K, Zhang H, Flanagan J G, Soriano P, Lu Q. Regulation of neural progenitor cell state by ephrin-B. The Journal of cell biology. 2008; 181(6):973-83. PubMed PMID: 18541704.
46. Qiu R., Wang J., Tsark W., and Lu Q. Essential role of PDZ-RGS3 in the maintenance of neural progenitor cells. Stem cells, 2010; 28:1602-1610.
47. Qiu, R., Wu, J., Gudenas, B., Northcott, P. A., Wechsler-Reya, R., and Lu, Q. Depletion of kinesin motor KIF20A to target cell fate control suppresses medulloblastoma tumour growth. Commun Biol4, 552 (2021). https://doi.org/10.1038/s42003-021-02075-4.

1. A method of treating cancer in a subject, comprising administering to the subject an effective amount of an RNA-based inhibitor or a peptide inhibitor which prevents or blocks binding of kinesin family member 20A (KIF20A) to regulator of G-protein signaling 3 (RGS3) or to septin-7 (SEPT7) or binding of EphrinB to RGS3.

2. A method of inhibiting proliferation of a daughter cell produced by a cancer cell in a subject, comprising administering to the subject an effective amount of an RNA-based inhibitor or a peptide inhibitor which targets one of KIF20A, SEPT7, RGS3 and EphrinB which prevents or blocks binding of KIF20A to RGS3 or to SEPT7 or binding of EphrinB to RGS3.

3. The method of claim 1, wherein the RNA-based inhibitor is an siRNA, an shRNA, or a miRNA targeting KIF20A, SEPT7, RGS3 or EphrinB.

4. The method of claim 1, wherein the peptide inhibitor is a peptide comprising an amino acid sequence at least 75%, at least 80%, at least 85%, at least 90%, at least 95%, at least 98%, at least 99%, or 100% identical to the amino acid sequence of human KIF20A residues 626-820, or a fragment thereof.

5. The method of claim 1, wherein the peptide inhibitor is a small peptide comprising 33 amino acid residues or less of the C-terminus of a conserved EphrinB protein.

6-10. (canceled)

11. The method of claim 1, wherein the peptide inhibitor is a peptide comprising an amino acid sequence at least 75%, at least 80%, at least 85%, at least 90%, at least 95%, at least 98%, at least 99%, or 100% identical to the amino acid sequence of the PDZ domain of mouse or human RGS3 isoform 1 (PDZ-RGS3) residues 18-94.

12. The method of claim 1, wherein the cancer includes brain tumor, leukemia, breast cancer, lung cancer, colon cancer, and liver cancer.

13. (canceled)

14. A composition for treating a cancer in a subject comprising an siRNA, an shRNA, or a miRNA targeting KIF20A, SEPT7, RGS3 or EphrinB, a peptide comprising an amino acid sequence at least 75%, at least 80%, at least 85%, at least 90%, at least 95%, at least 98%, at least 99%, or 100% identical to the amino acid sequence of mouse KIF20A residues 625-818 or human KIF20A residues 626-820, or a fragment thereof, a small peptide comprising 33 amino acid residues or less of the C-terminus of the conserved EphrinB proteins, or a peptide comprising an amino acid sequence at least 75%, at least 80%, at least 85%, at least 90%, at least 95%, at least 98%, at least 99%, or 100% identical to the amino acid sequence of the PDZ domain of mouse or human RGS3 isoform 1 (PDZ-RGS3) residues 18-94.

15-19. (canceled)

20. The composition of claim 14, wherein the siRNA, shRNA, miRNA, the peptide or a fragment thereof, or the small peptide is conjugated to a delivery vehicle.

21. The composition of claim 20, wherein the delivery vehicle is a nanoparticle.

22. The composition of claim 14, further comprising one or more pharmaceutically acceptable excipients or carriers.

23. The composition of claim 14, wherein the cancer includes brain tumor, leukemia, breast cancer, lung cancer, colon cancer, and liver cancer.

24. (canceled)

25. The method of claim 4, wherein the small peptide comprises an amino acid sequence at least 75%, at least 80%, at least 85%, at least 90%, at least 95%, at least 98%, at least 99%, or 100% identical to an amino acid sequence selected from the group consisting of CPHYEKVSGDYGHPVYIVQEMPPQSPANIYYKV (SEQ ID NO: 117), CPHYEKVSGDYGHPVYIVQDGPPQSPPNIYYKV (SEQ ID NO: 118), SPANIYYKV (SEQ ID NO: 1), ANIYYKV (SEQ ID NO: 2), and NIYYKV (SEQ ID NO: 3).

26. The method of claim 2, wherein the RNA-based inhibitor is an siRNA, an shRNA, or a miRNA targeting KIF20A, SEPT7, RGS3 or EphrinB.

27. The method of claim 2, wherein the peptide inhibitor is a peptide comprising an amino acid sequence at least 75%, at least 80%, at least 85%, at least 90%, at least 95%, at least 98%, at least 99%, or 100% identical to the amino acid sequence of human KIF20A residues 626-820, or a fragment thereof.

28. The method of claim 2, wherein the peptide inhibitor is a small peptide comprising 33 amino acid residues or less of the C-terminus of the conserved EphrinB proteins.

29. The method of claim 28, wherein the small peptide comprises an amino acid sequence at least 75%, at least 80%, at least 85%, at least 90%, at least 95%, at least 98%, at least 99%, or 100% identical to an amino acid sequence selected from the group consisting of CPHYEKVSGDYGHPVYIVQEMPPQSPANIYYKV (SEQ ID NO: 117), CPHYEKVSGDYGHPVYIVQDGPPQSPPNIYYKV (SEQ ID NO: 118), SPANIYYKV (SEQ ID NO: 1), ANIYYKV (SEQ ID NO: 2), and NIYYKV (SEQ ID NO: 3).

30. The method of claim 2, wherein the peptide inhibitor is a peptide comprising an amino acid sequence at least 75%, at least 80%, at least 85%, at least 90%, at least 95%, at least 98%, at least 99%, or 100% identical to the amino acid sequence of the PDZ domain of mouse or human RGS3 isoform 1 (PDZ-RGS3) residues 18-94.

31. The method of claim 2, wherein the cancer includes brain tumor, leukemia, breast cancer, lung cancer, colon cancer, and liver cancer.

32. The method of claim 14, wherein the small peptide comprises an amino acid sequence at least 75%, at least 80%, at least 85%, at least 90%, at least 95%, at least 98%, at least 99%, or 100% identical to an amino acid sequence selected from the group consisting of CPHYEKVSGDYGHPVYIVQEMPPQSPANIYYKV (SEQ ID NO: 117), CPHYEKVSGDYGHPVYIVQDGPPQSPPNIYYKV (SEQ ID NO: 118), SPANIYYKV (SEQ ID NO: 1), ANIYYKV (SEQ ID NO: 2), and NIYYKV (SEQ ID NO: 3).


SEQUENCE LISTING (128 sequences)

SEQ ID NO: 1 - protein, 9 aa, Artificial Sequence
  synthetic
SPANIYYKV

SEQ ID NO: 2 - protein, 7 aa, Artificial Sequence
  synthetic
ANIYYKV

SEQ ID NO: 3 - protein, 6 aa, Artificial Sequence
  synthetic
NIYYKV

SEQ ID NO: 4 - DNA, 2664 bp, Mus musculus
  misc_feature (1)..(3): start codon
  misc_feature (340)..(360): shRNA/siRNA targeting sequence
  misc_feature (429)..(449): shRNA/siRNA targeting sequence
  misc_feature (603)..(623): shRNA/siRNA targeting sequence
  misc_feature (629)..(650): shRNA/siRNA targeting sequence
  misc_feature (666)..(686): shRNA/siRNA targeting sequence
  misc_feature (1181)..(1201): shRNA/siRNA targeting sequence
  misc_feature (1775)..(1795): shRNA/siRNA targeting sequence
  misc_feature (1854)..(1874): shRNA/siRNA targeting sequence
  misc_feature (2360)..(2380): shRNA/siRNA targeting sequence
  misc_feature (2662)..(2664): stop codon
atgtctcaccggatcctttctccgccagcgggcttactctctgatgaggatgttgtagactctcccatattagaatccacagctgcagatctgagatctgttgtccgaaaggacctgttgtcagactgctctgtcatctctgcctctctggaggacaagcaggctctactggaggacacctcagagaaggtgaaagtttaccttcgaattcgaccattcttgacttcagagttggaccgacaggaagatcagggctgcgtctgcattgagaatacagaaacccttgtgctgcaggcacccaaagactcctttgccttgaagagtaatgagaggggagttggtcaggccactcacaaatttaccttttcccagatatttgggccagaagtgggacaggtggccttcttcaatctgaccatgaaggagatggtgaaggatgtgctcaaagggcagaactggctcatctacacctatggagtcaccaactcggggaaaacgtacaccattcaaggtactagtaaggatgcggggatcctgccccagtccctggctctcatctttaatagtctccagggccagcttcatccgacacctgacctgaagcccttattgtccaatgaagtaatctggctagacagcaagcagattcgacaggaggaaatgaagaagctgtccttgctaattggaggcctccaagaggaggagctgtccacctccgtgaagaaacgtgtccacactgaaagtcggataggtgccagcaacagctttgacagtggcgttgctgggctgtcttccaccagtcagttcaccagcagcagccagctagatgaaacaagtcaattatgggcacaaccagacactgtcccagtaagtgttccagcagacattcgcttctctgtctggatctccttctttgagatctacaatgaattgctttatgacctgttagaaccacctagccatcagcataagagacagacacttcggctgtgtgaggatcagaacggcaatccttacgtgaaagatctcaattggattcatgttcgtgatgttgaggaagcctggaaactattgaaagtgggtcgcaagaaccagagctttgccagcacccacatgaaccagcaatccagccgcagtcacagcatcttctcaatcagaatcttgcaccttcagggagaaggggatatagtgcccaagatcagtgaactgtcactctgtgacctggctggctctgagcgctgcaaacatcaaaaaagtggtgagcggctaaaggaggcagggaacattaacacttctctgcacaccctgggccgctgtattgctgccctgcgacagaatcagcagaaccggtcaaagcagaacctgattcctttccgtgacagcaagttgactcgtgtgttccaaggcttcttcacaggtcgaggtcgttcctgtatgattgtcaatgtgaatccctgtgcatctacatatgacgagactcttcatgcagccaaattctcagccctagccagccagcttgtgcacgccccacctgtgcatctgggaatcccatccctgcattcattcatcaagaagcacagtcctcaggttggccctggcttagagaaagaggacaaggccgactcagaccttgaggacagtcctgaagacgaagctgacgtctccgtgtatggcaaggaggaactactccaggtggtggaagccatgaaagcgctacttttaaaagaacgacaagaaaagctgcagttggagatacagctccgagaggaaatttgcaacgaaatggtggaacagatgcaacagcgggagcagtggtgcagtgagcgtttggacaaccaaaaggaactgatggaggaactgtacgaggagaaactgaagatcctgaaggaatcactgacaactttctaccaagagcagatccaggagcgggatgaaaaaattgaagagctagaaactctgttgcaggaagccaaacagcagccagcggcacaacagtcaggggggctgtccctcctccggcggtcacagaggctggcagcgtctgcctccactcagcagttccaggaagttaaagctgaactagagcaatgcaaaacagagttaagctctaccactgcagagctgcacaagtatcagcaagtgttaaaaccgccacccccagccaagcctttcaccattgatgtggataagaagttagaggagggccagaagaatataagactgctacggacagagctccagaaactaggacagtctctgcagtcagcagaaagagcctgttgccacagcaccggagcaggaaaacttcgtcaagcattaaccaactgtgatgacatcttaatcaagcagaaccagaccctggctgagctgcagaataacatggtgctagtgaaactggaccttcagaaaaaggcagcctgcattgctgagcagtatcacactgtgctaaagctccaaggccaagcctctgccaaaaagcggctgggagcaaaccaggaaaaccagcaaccaaatcatcaacccccaggaaagaaaccattcctgcgaaacttacttccccgaacacctacctgccaaagctcaacagacagcagcccttatgcgcggatcttgcgctcacggcactctcctttactcaaatctccctttggcaaaaaatactaa

SEQ ID NO: 5 - DNA, 662 bp, Mus musculus
  misc_feature (9)..(29): shRNA/siRNA targeting sequence
agttgtggggatgaagatgcatggttgttgccctgaggttggtcagctgcttagctgataagtctcttttaagcttcagcatacacccagaactgtattcagaattattgtattataatcacctatgtaatcttgttttttttttcatgcacaaatattatatggaataaagacattgtttatgcttttatttggattccaaatttagaaaaatataaaaagggggacagaaaattgatataaataccatctggcctgttagacccttggacaggaacagcagtgggaaccagcctaatgcacactaggaaatgagtacaattttaaatgatacccaagaggcttgggtagctttgtctggccaggaagttgctatgtcaaaacacccacctgcctgtctccaaatgggattaaagacatgcaccaccacatggcacccagttagtgttccttccattaaatggggagtaaaatgttctgagtacaagagaaggaaggctagctcctaaccacagaatgatctatagcacctactgcacaagctcattgtagcagacatcaagtggctctgaaacctagaaaaacacagtgaattcttcaaccttgtagataaggttgaaaaccaaattggagactttaaaatccgaagtaaaaaagcttcc

SEQ ID NO: 6 - DNA, 2673 bp, Homo sapiens
  misc_feature (1)..(3): start codon
  misc_feature (181)..(201): shRNA/siRNA targeting sequence
  misc_feature (400)..(420): shRNA/siRNA targeting sequence
  misc_feature (726)..(746): shRNA/siRNA targeting sequence
  misc_feature (905)..(925): shRNA/siRNA targeting sequence
  misc_feature (1462)..(1482): shRNA/siRNA targeting sequence
  misc_feature (1752)..(1772): shRNA/siRNA targeting sequence
  misc_feature (1809)..(1829): shRNA/siRNA targeting sequence
  misc_feature (2061)..(2081): shRNA/siRNA targeting sequence
  misc_feature (2091)..(2111): shRNA/siRNA targeting sequence
  misc_feature (2226)..(2246): shRNA/siRNA targeting sequence
  misc_feature (2376)..(2396): shRNA/siRNA targeting sequence
  misc_feature (2671)..(2673): stop codon
atgtcgcaagggatcctttctccgccagcgggcttgctgtccgatgacgatgtcgtagtttctcccatgtttgagtccacagctgcagatttggggtctgtggtacgcaagaacctgctatcagactgctctgtcgtctctacctccctagaggacaagcagcaggttccatctgaggacagtatggagaaggtgaaagtatacttgagggttaggcccttgttaccttcagagttggaacgacaggaagatcagggttgtgtccgtattgagaatgtggagacccttgttctacaagcacccaaggactcttttgccctgaagagcaatgaacggggaattggccaagccacacacaggttcaccttttcccagatctttgggccagaagtgggacaggcatccttcttcaacctaactgtgaaggagatggtaaaggatgtactcaaagggcagaactggctcatctatacatatggagtcactaactcagggaaaacccacacgattcaaggtaccatcaaggatggagggattctcccccggtccctggcgctgatcttcaatagcctccaaggccaacttcatccaacacctgatctgaagcccttgctctccaatgaggtaatctggctagacagcaagcagatccgacaggaggaaatgaagaagctgtccctgctaaatggaggcctccaagaggaggagctgtccacttccttgaagaggagtgtctacatcgaaagtcggataggtaccagcaccagcttcgacagtggcattgctgggctctcttctatcagtcagtgtaccagcagtagccagctggatgaaacaagtcatcgatgggcacagccagacactgccccactacctgtcccggcaaacattcgcttctccatctggatctcattctttgagatctacaacgaactgctttatgacctattagaaccgcctagccaacagcgcaagaggcagactttgcggctatgcgaggatcaaaatggcaatccctatgtgaaagatctcaactggattcatgtgcaagatgctgaggaggcctggaagctcctaaaagtgggtcgtaagaaccagagctttgccagcacccacctcaaccagaactccagccgcagtcacagcatcttctcaatcaggatcctacaccttcagggggaaggagatatagtccccaagatcagcgagctgtcactctgtgatctggctggctcagagcgctgcaaagatcagaagagtggtgaacggttgaaggaagcaggaaacattaacacctctctacacaccctgggccgctgtattgctgcccttcgtcaaaaccagcagaaccggtcaaagcagaacctggttcccttccgtgacagcaagttgactcgagtgttccaaggtttcttcacaggccgaggccgttcctgcatgattgtcaatgtgaatccctgtgcatctacctatgatgaaactcttcatgtggccaagttctcagccattgctagccagcttgtgcatgccccacctatgcaactgggattcccatccctgcactcgttcatcaaggaacatagtcttcaggtatcccccagcttagagaaaggggctaaggcagacacaggccttgatgatgatattgaaaatgaagctgacatctccatgtatggcaaagaggagctcctacaagttgtggaagccatgaagacactgcttttgaaggaacgacaggaaaagctacagctggagatgcatctccgagatgaaatttgcaatgagatggtagaacagatgcaacagcgggaacagtggtgcagtgaacatttggacacccaaaaggaactattggaggaaatgtatgaagaaaaactaaatatcctcaaggagtcactgacaagtttttaccaagaagagattcaggagcgggatgaaaagattgaagagctagaagctctcttgcaggaagccagacaacagtcagtggcccatcagcaatcagggtctgaattggccctacggcggtcacaaaggttggcagcttctgcctccacccagcagcttcaggaggttaaagctaaattacagcagtgcaaagcagagctaaactctaccactgaagagttgcataagtatcagaaaatgttagaaccaccaccctcagccaagcccttcaccattgatgtggacaagaagttagaagagggccagaagaatataaggctgttgcggacagagcttcagaaacttggtgagtctctccaatcagcagagagagcttgttgccacagcactggggcaggaaaacttcgtcaagccttgaccacttgtgatgacatcttaatcaaacaggaccagactctggctgaactgcagaacaacatggtgctagtgaaactggaccttcggaagaaggcagcatgtattgctgagcagtatcatactgtgttgaaactccaaggccaggtttctgccaaaaagcgccttggtaccaaccaggaaaatcagcaaccaaaccaacaaccaccagggaagaaaccattccttcgaaatttacttccccgaacaccaacctgccaaagctcaacagactgcagcccttatgcccggatcctacgctcacggcgttcccctttactcaaatctgggccttttggcaaaaagtactaa

SEQ ID NO: 7 - DNA, 1038 bp, Mus musculus
  misc_feature (1)..(3): start codon
  misc_feature (14)..(49): shRNA/siRNA targeting sequence
  misc_feature (110)..(130): shRNA/siRNA targeting sequence
  misc_feature (150)..(170): shRNA/siRNA targeting sequence
  misc_feature (212)..(232): shRNA/siRNA targeting sequence
  misc_feature (269)..(289): shRNA/siRNA targeting sequence
  misc_feature (331)..(351): shRNA/siRNA targeting sequence
  misc_feature (467)..(487): shRNA/siRNA targeting sequence
  misc_feature (1010)..(1030): shRNA/siRNA targeting sequence
  misc_feature (1036)..(1038): stop codon
atggcccggcctgggcagcgttggctcagcaagtggcttgtggctatggtcgtgctgacgctgtgccggcttgccacgccgttggccaagaacctggagcccgtgtcctggagctctcttaaccctaagttcctaagtgggaagggcttggtgatctacccgaagattggagacaagctggacatcatctgcccccgagcagaagcagggcggccctacgagtactacaagctgtacctggtgcggccagagcaggcggctgcttgcagcactgtgcttgatcccaatgtactggtcacttgcaacaagccacaccaggaaatccgcttcaccatcaagttccaagagttcagccccaactacatgggcctggaattcaaaaagtaccacgattactacattacatcaacgtccaatgggagcttggagggactggagaaccgggagggaggtgtgtgtcgcacccgcactatgaagatcgttatgaaggttgggcaagatccaaatgctgtgacacccgagcagttgactaccagccggccaagcaaggagtcagacaacactgtcaagacagccacacaggctcctggtcggggatcccagggtgactctgacggcaagcatgagactgtgaaccaggaagagaagagtggcccaggtgcaggtggcggtggcagcggggactctgacagcttcttcaactccaaggtagcattgttcgcagccgtcggcgccggctgtgtcatcttcctgctcatcatcatcttcttgacagtcctactactcaagctccgcaagcgccatcgcaagcatacacagcagcgggcggctgccctctcgctcagtaccctagccagccccaaagggggtagtggtacagcgggcaccgagcccagcgacatcatcatccccttacggactacagagaacaactactgcccccactatgagaaggtgagtggggactacgggcatcctgtctacatcgtccaggagatgccccctcagagcccggcgaacatctactacaaggtttga

SEQ ID NO: 8 - DNA, 718 bp, Mus musculus
  misc_feature (334)..(354): shRNA/siRNA targeting sequence
  misc_feature (587)..(607): shRNA/siRNA targeting sequence
  misc_feature (679)..(699): shRNA/siRNA targeting sequence
aggcccagcatggcctcaggccccacaggacacatggcctggatcggccgctctcttctcccccacacccctgcccttgccagctgtgcccacctttgtatttagttttgtagtttcttggcttttataatcccctttttcttgccccctgggcttcggaggggggtgcttgtgcccccaatccccatgctcttgtgccttccccctctggccaggcctctgggctccgtgggggtgccctttctaggagggcagggttggacactgacggactgcaggcagggaggtagccccctggccctgccccctcctcatcccttcacccatccccaggactgctcgtccactatcatcactgtttttaatgcttttgtgttgattttttttaaaagctgtcaactcattttcatctgtttttttttttttgagaaaaaaaaatgggaaaatgtacaaggcagccccttcccaggctttctgagcctggcctgacccagtctaagagggcctggggcaggatgtggccagccaggaaacataggatggcatttcttttatagactccttagtagttcaggggagtgggggagggtaggccagggctatttctgcccaccgaggagagaagagcgccaccctcctttttggaccctcatgagacaatgctgttcctagcagtggggtagtgactaccacccttctaccaaaaaaaaaaaaaaag

SEQ ID NO: 9 - DNA, 2793 bp, Mus musculus
  misc_feature (1)..(3): start codon
  misc_feature (8)..(28): shRNA/siRNA targeting sequence
  misc_feature (1185)..(1205): shRNA/siRNA targeting sequence
  misc_feature (1469)..(1489): shRNA/siRNA targeting sequence
  misc_feature (1555)..(1575): shRNA/siRNA targeting sequence
  misc_feature (1749)..(1769): shRNA/siRNA targeting sequence
  misc_feature (1785)..(1805): shRNA/siRNA targeting sequence
  misc_feature (2221)..(2241): shRNA/siRNA targeting sequence
  misc_feature (2245)..(2265): shRNA/siRNA targeting sequence
  misc_feature (2278)..(2298): shRNA/siRNA targeting sequence
  misc_feature (2791)..(2793): stop codon
atgaaccgcttcaatgggctctgcaaagtgtgttcagaacgccgctaccggcagatcaccatccggaggggcaaagacggctttggcttcaccatctgctgtgactctccggtccgagtccaggctgtggattctgggggcccggcagagagggcgggactgcagcagctggacacagtgctacaactgaatgagagacccgtggagcactggaaatgtgtggagctggcacatgagatccggagctgtcctagcgagatcatcctgctcgtgtggcgtgtggtcccccagatcaagccggggccagatggcggagtcttgcggcgggcctcctgcaagtccacacatgacctcctgtcaccccctaacaagagggagaagaactgtactcatggggccccagttcgtcctgagcagcgccacagctgccacctggtgtgtgacagctctgatggtctactgcttggtggctgggagcgctacactgaggtgggcaagcgcagtggccagcacaccctgcctgcactgtcccggaccaccacccctactgaccccaactacatcatcctggccccactgaatcctggaagccagttgctgcggcctgtgtaccaggaggatacaatccctgaagaaccggggactactactaaagggaaatcgtacaccggcctgggcaagaagtctcggctcatgaagacagtgcagaccatgaagggccacagtaactaccaagactgctcagccctgagaccgcacatcccgcattccagttacggcacctatgtcaccctggcccctaaagtcctggtgttccctgtctttgtgcagcccctagatctctgtaaccctgcccggactctcctgctgtcggaggagctgctgctgtatgaggggaggaacaagacttcccaggtgacactgtttgcctactcggacctgctgctgttcactaaggaggaggagccaggccgctgcgacgtcctgagaaatcccctctacctccagagcgtgaagctacaggagggctcttcagaagacttgaaattctgtgtgctgtacctggcagagaaggcagagtgcttattcactttggaggcacactcgcaggagcagaagaagagagtgtgctggtgcctgtcggagaacatcgccaagcagcaacagctggccgcaccacctacagagaggaagatgtttgagacagaggcagatgagaaggagatgcccctggtcgaggggaaggggccaggtgctgaggaacccgcacccagcaaaaatccctctcctggccaggagcttcctccaggacaagacctccctcccagcaaagacccctctcccagccaggagcttcctgcaggacaagatctcccacccaggaaggactccccaggccaagaagctgctcctggcccagaatcaccatccagtgaagacatagcaacctgccctaagccccctcaaagcccagaaacctcaacaagcaaggactccccaccaggccagggatcctccccgaccacagaactcccatcttgccagggccttcctgctggtcaagaatctactagccaggaccctctgctcagtcaagagccccctgttatcccagaatcctctgcctccgtccagaaacgcctaccctctcaggagtcaccctccagcctgggctccctgccagagaaggaccttgctgagcagaccatcagctccggggagccaccagtcgccactggtgctgtactgccagcctctagacctaactttgtgatccccgaggtgcgtctggataatgcctacagccaactggatggggcccacggaggcagctctggcgaggacgaggatgcagaagagggagaggaggggggagaaggggaggaagatgaggaggacgacaccagcgacgacaactacggggatcgtagtgaggccaagcgcagcagcctgattgagactggccaaggcgccgagggcggcttctcgttgcgtgtgcagaactcgctgcggcgccggacgcacagcgagggcagcctgctgcaggagtcccgggggccctgctttgcctctgacaccaccttacactgctctgatggcgagggcgccacatccacctgggctatcccttcaccccgcaccctcaaaaaagaactgggtcgtaatggaggctccatgcaccacctttccctgttcttcacgggacacaggaagatgagtgggactgacctcacagaatgtgatgaagcttcccggaagagaaagagcaaaaacatagccaaggacatgaagaataagctggccatcttcaggcggcggaacgaatctcccggggctcagccagccagcaagacagacaagacaaccaagtccttcaagcctacctcggaggaagccctcaagtggagcgagtccctggaaaagttgctgcttcataaatatggcttagaagtgttccaggccttccttcgcaccgagttcagtgaggagaacctggagttttggctcgcttgcgaggacttcaagaaggtcaaatcacagtccaagatggcagccaaagccaagaagatctttgctgaattcatcgcaatccaggcttgcaaagaggtaaacctggactcctacacacgagaacacaccaaggagaatctgcagagcatcacccgaggctgctttgacttggcacagaaacgtatctttgggctcatggagaaggactcttaccctcgcttcctccgctctgacctctacctggacctcattaaccagaagaagatgagtcccccgctctag

SEQ ID NO: 10 - DNA, 1311 bp, Mus musculus
  misc_feature (1)..(3): start codon
  misc_feature (705)..(729): shRNA/siRNA targeting sequence
  misc_feature (736)..(756): shRNA/siRNA targeting sequence
  misc_feature (879)..(904): shRNA/siRNA targeting sequence
  misc_feature (1069)..(1093): shRNA/siRNA targeting sequence
  misc_feature (1258)..(1282): shRNA/siRNA targeting sequence
  misc_feature (1309)..(1311): stop codon
atgtcggtcagtgcgagatccgctgctgccgaggagaggagcgtcaactgcggcaccatggctcaaccgaagaaccttgagggctatgtgggctttgccaacctcccaaatcaagtgtacagaaaatccgtgaaaagaggatttgaattcactcttatggtagtaggtgaatctggactgggaaagtcgacattaatcaactcattattcctcacagatttgtattctccagagtatccaggaccttctcatagaatcaaaaagactgtacaggtggagcaatccaaagttttaatcaaagaaggtggtgttcagttgctgctgacaatagttgatactccaggatttggagatgcagtggataatagtaattgctggcagcctgttatcgactacattgatagtaaatttgaagattacttaaatgcagaatctcgagtgaacagacgtcagatgcctgataacagggtgcagtgttgtttatacttcattgctccttcaggacatggacttaaaccattggacattgagtttatgaaacgtttgcatgaaaaagtgaatatcatcccattaattgccaaagcagacacacttacaccagaggaatgccaacagtttaaaaagcagataatgaaagaaattcaagaacataaaattaaaatatatgaatttccagaaacagatgatgaagaagagaataaactggttaagaagataaaggaccgtttacctcttgctgtggtaggtagtaatactatcattgaagttaatggcaaaagagtcagaggaaggcagtatccttggggtgtggctgaagttgaaaatggtgaacattgtgatttcacaattctaagaaatatgttgataagaacacatatgcaggacttgaaagatgttaccaataacgtacactatgagaactacagaagcagaaagctagcagcagtgacttacaatggagtggataacaacaagaacaaagggcagcttaccaagagccctctggcacagatggaagaagaaagaagggaacatgtagccaaaatgaagaagatggagatggagatggaacaggtgtttgagatgaaggtcaaagaaaaagttcaaaaactgaaggactctgaagcagagctccagcggcgccatgagcaaatgaaaaagaatttagaagcacagcacaaagaattagaggaaaaacgtcgtcagtttgaagaagaaaaggcaaactgggaagctcaacagcgtattttagagcaacagaactcttcaagaaccttggaaaagaacaagaagaaaggcaagatcttttaa

SEQ ID NO: 11 - DNA, 1014 bp, Mus musculus
  misc_feature (48)..(72): shRNA/siRNA targeting sequence
  misc_feature (162)..(183): shRNA/siRNA targeting sequence
  misc_feature (425)..(449): shRNA/siRNA targeting sequence
  misc_feature (840)..(864): shRNA/siRNA targeting sequence
  misc_feature (917)..(941): shRNA/siRNA targeting sequence
actctattgaccaccagttatgtattagttgccaatatgccagcttggacatcagtgtttgttggatccgtttgaccaatttgcaccaattttatccataatgatggatttaacagcatgacaaaaattattttttgttgttctcgaaggagattaagatgccttgaattgtctaggatattgtgtacttagaaattaacagctctaagtacctttctacattttttttcttttttttttaaattaaaagatgtcttcagtttaatgcaagaaaacattttactgttgtacaatcatgttctggtggtttgattgtttacagaatattctaaaataaaaggactctggaaggttttcattgaggataaattgccataatatgatgcaaactatgcttctctatgataattataatacagaggttccattcgatgcagcctatacaataatgtatttagtctaacacagtggaccctattttttgacacttccattgtttaaaagtacacatggaaaaaacatatatgcttacagtgcacctagagctttttataacagccttttttgtttgtttgtttgttttggattctttaaatatataaatatattattctcatttagtgcccctgtagccagaacctcattactgcttcatttttgtaataacatttaatttagatattttccatatattggccctgctaaaatagaatatagcatctttcatatggtaggaaccaacgaggaaactttcctttaactccctttttacactttatggaaagtagcaggaggagaaatgcatttgtagatcatttctaggcaaattgtgaagctaacgaccagcctgtttcttcctatactcagtcttgttttactagaaatgggaatcatggcctcttgaagagaaaaaagtcaccattctgcatttagctgtatctatatattgcatacctgtatttttttgtttgtattgtaaaaaaaattcacataataaacaatgttgtgatgtaa

SEQ ID NO: 12 - DNA, 21 bp, Mus musculus
ggtcaggccactcacaaattt

SEQ ID NO: 13 - RNA, 21 nt, Mus musculus
ggucaggccacucacaaauuu

SEQ ID NO: 14 - DNA, 21 bp, Mus musculus
ggtgaaggatgtgctcaaagg

SEQ ID NO: 15 - RNA, 21 nt, Mus musculus
ggugaaggaugugcucaaagg

SEQ ID NO: 16 - DNA, 21 bp, Mus musculus
gcccttattgtccaatgaagt

SEQ ID NO: 17 - RNA, 21 nt, Mus musculus
gcccuuauuguccaaugaagu

SEQ ID NO: 18 - DNA, 22 bp, Mus musculus
ggctagacagcaagcagattcg

SEQ ID NO: 19 - RNA, 22 nt, Mus musculus
ggcuagacagcaagcagauucg

SEQ ID NO: 20 - DNA, 20 bp, Mus musculus
aagctgtccttgctaattgg

SEQ ID NO: 21 - RNA, 21 nt, Mus musculus
gaagcuguccuugcuaauugg

SEQ ID NO: 22 - DNA, 21 bp, Mus musculus
gggatatagtgcccaagatca

SEQ ID NO: 23 - RNA, 21 nt, Mus musculus
gggauauagugcccaagauca

SEQ ID NO: 24 - DNA, 21 bp, Mus musculus
gcaacgaaatggtggaacaga

SEQ ID NO: 25 - RNA, 21 nt, Mus musculus
gcaacgaaaugguggaacaga

SEQ ID NO: 26 - DNA, 21 bp, Mus musculus
ggaactgtacgaggagaaact

SEQ ID NO: 27 - RNA, 21 nt, Mus musculus
ggaacuguacgaggagaaacu

SEQ ID NO: 28 - DNA, 21 bp, Mus musculus
tggctgagctgcagaataaca

SEQ ID NO: 29 - RNA, 21 nt, Mus musculus
uggcugagcugcagaauaaca

SEQ ID NO: 30 - DNA, 21 bp, Mus musculus
ggatgaagatgcatggttgtt

SEQ ID NO: 31 - RNA, 21 nt, Mus musculus
ggaugaagaugcaugguuguu

SEQ ID NO: 32 - DNA, 21 bp, Homo sapiens
agtatggagaaggtgaaagta

SEQ ID NO: 33 - RNA, 21 nt, Homo sapiens
aguauggagaaggugaaagua

SEQ ID NO: 34 - DNA, 21 bp, Homo sapiens
gcatccttcttcaacctaact

SEQ ID NO: 35 - RNA, 21 nt, Homo sapiens
gcauccuucuucaaccuaacu

SEQ ID NO: 36 - DNA, 21 bp, Homo sapiens
gaggagtgtctacatcgaaag

SEQ ID NO: 37 - RNA, 21 nt, Homo sapiens
gaggagugucuacaucgaaag

SEQ ID NO: 38 - DNA, 21 bp, Homo sapiens
ggatctcattctttgagatct

SEQ ID NO: 39 - RNA, 21 nt, Homo sapiens
ggaucucauucuuugagaucu

SEQ ID NO: 40 - DNA, 21 bp, Homo sapiens
gcatctacctatgatgaaact

SEQ ID NO: 41 - RNA, 21 nt, Homo sapiens
gcaucuaccuaugaugaaacu

SEQ ID NO: 42 - DNA, 21 bp, Homo sapiens
ggagatgcatctccgagatga

SEQ ID NO: 43 - RNA, 21 nt, Homo sapiens
ggagaugcaucuccgagauga

SEQ ID NO: 44 - DNA, 21 bp, Homo sapiens
ggaacagtggtgcagtgaaca

SEQ ID NO: 45 - RNA, 21 nt, Homo sapiens
ggaacaguggugcagugaaca

SEQ ID NO: 46 - DNA, 21 bp, Homo sapiens
gcagcttcaggaggttaaagc

SEQ ID NO: 47 - RNA, 21 nt, Homo sapiens
gcagcuucaggagguuaaagc

SEQ ID NO: 48 - DNA, 21 bp, Homo sapiens
gcagtgcaaagcagagctaaa

SEQ ID NO: 49 - RNA, 21 nt, Homo sapiens
gcagugcaaagcagagcuaaa

SEQ ID NO: 50 - DNA, 21 bp, Homo sapiens
gctgttgcggacagagcttca

SEQ ID NO: 51 - RNA, 21 nt, Homo sapiens
gcuguugcggacagagcuuca

SEQ ID NO: 52 - DNA, 21 bp, Homo sapiens
gcagaacaacatggtgctagt

SEQ ID NO: 53 - RNA, 21 nt, Homo sapiens
gcagaacaacauggugcuagu

SEQ ID NO: 54 - DNA, 21 bp, Mus musculus
ggcagcgttggctcagcaagt

SEQ ID NO: 55 - RNA, 21 nt, Mus musculus
ggcagcguuggcucagcaagu

SEQ ID NO: 56 - DNA, 21 bp, Mus musculus
gcaagtggcttgtggctatgg

SEQ ID NO: 57 - RNA, 21 nt, Mus musculus
gcaaguggcuuguggcuaugg

SEQ ID NO: 58 - DNA, 21 bp, Mus musculus
ggagctctcttaaccctaagt

SEQ ID NO: 59 - RNA, 21 nt, Mus musculus
ggagcucucuuaacccuaagu

SEQ ID NO: 60 - DNA, 21 bp, Mus musculus
ggtgatctacccgaagattgg

SEQ ID NO: 61 - RNA, 21 nt, Mus musculus
ggugaucuacccgaagauugg

SEQ ID NO: 62 - DNA, 21 bp, Mus musculus
ggccctacgagtactacaagc

SEQ ID NO: 63 - RNA, 21 nt, Mus musculus
ggcccuacgaguacuacaagc

SEQ ID NO: 64 - DNA, 21 bp, Mus musculus
gcactgtgcttgatcccaatg

SEQ ID NO: 65 - RNA, 21 nt, Mus musculus
gcacugugcuugaucccaaug

SEQ ID NO: 66 - DNA, 21 bp, Mus musculus
accatcaagttccaagagttc

SEQ ID NO: 67 - RNA, 21 nt, Mus musculus
accaucaaguuccaagaguuc

SEQ ID NO: 68 - DNA, 21 bp, Mus musculus
gcactatgaagatcgttatga

SEQ ID NO: 69 - RNA, 21 nt, Mus musculus
gcacuaugaagaucguuauga

SEQ ID NO: 70 - DNA, 21 bp, Mus musculus
gcccggcgaacatctactaca

SEQ ID NO: 71 - RNA, 21 nt, Mus musculus
gcccggcgaacaucuacuaca

SEQ ID NO: 72 - DNA, 21 bp, Mus musculus
gactgctcgtccactatcatc

SEQ ID NO: 73 - RNA, 21 nt, Mus musculus
gacugcucguccacuaucauc

SEQ ID NO: 74 - DNA, 21 bp, Mus musculus
gggtaggccagggctatttct

SEQ ID NO: 75 - RNA, 21 nt, Mus musculus
ggguaggccagggcuauuucu

SEQ ID NO: 76 - DNA, 21 bp, Mus musculus
ggtagtgactaccacccttct

SEQ ID NO: 77 - RNA, 21 nt, Mus musculus
gguagugacuaccacccuucu

SEQ ID NO: 78 - DNA, 21 bp, Mus musculus
gcttcaatgggctctgcaaag

SEQ ID NO: 79 - RNA, 21 nt, Mus musculus
gcuucaaugggcucugcaaag

SEQ ID NO: 80 - DNA, 21 bp, Mus musculus
agagaggaagatgtttgagac

SEQ ID NO: 81 - RNA, 21 nt, Mus musculus
agagaggaagauguuugagac

SEQ ID NO: 82 - DNA, 21 bp, Mus musculus
gcccagaaacctcaacaagca

SEQ ID NO: 83 - RNA, 21 nt, Mus musculus
gcccagaaaccucaacaagca

SEQ ID NO: 84 - DNA, 21 bp, Mus musculus
gctggtcaagaatctactagc

SEQ ID NO: 85 - RNA, 21 nt, Mus musculus
gcuggucaagaaucuacuagc

SEQ ID NO: 86 - DNA, 21 bp, Mus musculus
gccagcctctagacctaactt

SEQ ID NO: 87 - RNA, 21 nt, Mus musculus
gccagccucuagaccuaacuu

SEQ ID NO: 88 - DNA, 21 bp, Mus musculus
gcgtctggataatgcctacag

SEQ ID NO: 89 - RNA, 21 nt, Mus musculus
gcgucuggauaaugccuacag

SEQ ID NO: 90 - DNA, 21 bp, Mus musculus
gggactgacctcacagaatgt

SEQ ID NO: 91 - RNA, 21 nt, Mus musculus
gggacugaccucacagaaugu

SEQ ID NO: 92 - DNA, 21 bp, Mus musculus
gaagcttcccggaagagaaag

SEQ ID NO: 93 - RNA, 21 nt, Mus musculus
gaagcuucccggaagagaaag

SEQ ID NO: 94 - DNA, 21 bp, Mus musculus
gccaaggacatgaagaataag

SEQ ID NO: 95 - RNA, 21 nt, Mus musculus
gccaaggacaugaagaauaag

SEQ ID NO: 96 - DNA, 25 bp, Mus musculus
ggttaagaagataaaggaccgttta

SEQ ID NO: 97 - RNA, 25 nt, Mus musculus
gguuaagaagauaaaggaccguuua

SEQ ID NO: 98 - DNA, 21 bp, Mus musculus
gctgtggtaggtagtaatact

SEQ ID NO: 99 - RNA, 21 nt, Mus musculus
gcugugguagguaguaauacu

SEQ ID NO: 100 - DNA, 25 bp, Mus musculus
caggacttgaaagatgttaccaata

SEQ ID NO: 101 - RNA, 25 nt, Mus musculus
caggacuugaaagauguuaccaaua

SEQ ID NO: 102 - DNA, 25 bp, Mus musculus
gaacaggtgtttgagatgaaggtca

SEQ ID NO: 103 - RNA, 25 nt, Mus musculus
gaacagguguuugagaugaagguca

SEQ ID NO: 104 - DNA, 25 bp, Mus musculus
cagaactcttcaagaaccttggaaa

SEQ ID NO: 105 - RNA, 25 nt, Mus musculus
cagaacucuucaagaaccuuggaaa

SEQ ID NO: 106 - DNA, 25 bp, Mus musculus
gacatcagtgtttgttggatccgtt

SEQ ID NO: 107 - RNA, 25 nt, Mus musculus
gacaucaguguuuguuggauccguu

SEQ ID NO: 108 - DNA, 21 bp, Mus musculus
cttgaattgtctaggatattg

SEQ ID NO: 109 - RNA, 21 nt, Mus musculus
cuugaauugucuaggauauug

SEQ ID NO: 110 - DNA, 25 bp, Mus musculus
ccattcgatgcagcctatacaataa

SEQ ID NO: 111 - RNA, 25 nt, Mus musculus
ccauucgaugcagccuauacaauaa

SEQ ID NO: 112 - DNA, 25 bp, Mus musculus
cgaccagcctgtttcttcctatact

SEQ ID NO: 113 - RNA, 25 nt, Mus musculus
cgaccagccuguuucuuccuauacu

SEQ ID NO: 114 - DNA, 25 bp, Mus musculus
caccattctgcatttagctgtatct

SEQ ID NO: 115 - RNA, 25 nt, Mus musculus
caccauucugcauuuagcuguaucu

SEQ ID NO: 116 - protein, 195 aa, Homo sapiens
LNILKESLTSFYQEEIQERDEKIEELEALLQEARQQSVAHQQSGSELALRRSQRLAASASTQQLQEVKAKLQQCKAELNSTTEELHKYQKMLEPPPSAKPFTIDVDKKLEEGQKNIRLLRTELQKLGESLQSAERACCHSTGAGKLRQALTTCDDILIKQDQTLAELQNNMVLVKLDLRKKAACIAEQYHTVLKL

SEQ ID NO: 117 - protein, 33 aa, Artificial Sequence
  synthetic
CPHYEKVSGDYGHPVYIVQEMPPQSPANIYYKV

SEQ ID NO: 118 - protein, 33 aa, Artificial Sequence
  synthetic
CPHYEKVSGDYGHPVYIVQDGPPQSPPNIYYKV

SEQ ID NO: 119 - protein, 77 aa, Homo sapiens
QITIPRGKDGFGFTICCDSPVRVQAVDSGGPAERAGLQQLDTVLQLNERPVEHWKCVELAHEIRSCPSEIILLVWRM

SEQ ID NO: 120 - DNA, 47 bp, Artificial Sequence
  synthetic
  misc_feature (20)..(28): loop
cggctgaaacaagagttggttcaagagaccaactcttgtttcagccg

SEQ ID NO: 121 - DNA, 47 bp, Artificial Sequence
  synthetic
  misc_feature (20)..(28): loop
gaggagtgtctacatcgaattcaagagattcgatgtagacactcctc

SEQ ID NO: 122 - DNA, 47 bp, Artificial Sequence
  synthetic
  misc_feature (20)..(28): loop
agtatggagaaggtgaaagttcaagagactttcaccttctccatact

SEQ ID NO: 123 - protein, 194 aa, Mus musculus
LKILKESLTTFYQEQIQERDEKIEELETLLQEAKQQPAAQQSGGLSLLRRSQRLAASASTQQFQEVKAELEQCKTELSSTTAELHKYQQVLKPPPPAKPFTIDVDKKLEEGQKNIRLLRTELQKLGQSLQSAERACCHSTGAGKLRQALTNCDDILIKQNQTLAELQNNMVLVKLDLQKKAACIAEQYHTVLKL

SEQ ID NO: 124 - protein, 195 aa, Homo sapiens; identical to SEQ ID NO: 116

SEQ ID NO: 125 - protein, 83 aa, Artificial Sequence
  synthetic
KYRRRHRKHSPQHTTTLSLSTLATPKRGGNNNGSEPSDVIIPLRTADSVFCPHYEKVSGDYGHPVYIVQEMPPQSPANIYYKV

SEQ ID NO: 126 - protein, 83 aa, Artificial Sequence
  synthetic
KLRKRHRKHTQQRAAALSLSTLASPKGGSGTAGTEPSDIIIPLRTTENNYCPHYEKVSGDYGHPVYIVQEMPPQSPANIYYKV

SEQ ID NO: 127 - protein, 77 aa, Mus musculus
QITIRRGKDGFGFTICCDSPVRVQAVDSGGPAERAGLQQLDTVLQLNERPVEHWKCVELAHEIRSCPSEIILLVWRV

SEQ ID NO: 128 - protein, 77 aa, Homo sapiens; identical to SEQ ID NO: 119